(19) United States
(12) Patent Application Publication
(10) Pub. No.: US 2016/0184599 A1
(43) Pub. Date: Jun. 30, 2016

(54) REGENERATION OF NERVE FIBERS

(71) Applicant: BRAINQ TECHNOLOGIES LTD, Jerusalem (IL)

(72) Inventor: Yaron SEGAL, Jerusalem (IL)

(73) Assignee: BRAINQ TECHNOLOGIES LTD, Jerusalem (IL)

(21) Appl. No.: 14/911,488

(22) PCT Filed: Aug. 14, 2013
(86) PCT No.: PCT/IL13/50693

§ 371 Date: Feb. 11, 2016

(51) Int. Cl. A61N 2/00; A61B 5/04; A61N 2/02

(52) U.S. Cl. CPC A61N 2/006; A61N 2/02; A61B 5/04008

(57) ABSTRACT

The present invention discloses a method for regenerating nerve fibers of a subject comprising the steps of: (a) selecting at least a target portion of the nervous system of a subject with a lesion in the nervous system; (b) detecting electromagnetic wave profile of said at least a target portion of said nervous system of said subject; (c) comparing wave profiles of said subject with wave profiles of normal patients; and (d) defining a treatment protocol. According to a certain aspect, the step of defining said treatment protocol includes selecting compensating electromagnetic waves adapted for correcting anomalous wave profiles associated with said nervous system lesions and transmitting to said subject said protocol of compensating electromagnetic waves to provide a resonance effect; thereby inducing regeneration of nerve fibers at said nervous system lesions and regions proximate to said lesions. The present invention further discloses apparatus and a computer implemented method thereof.

### FIELD OF THE INNOVATION

This invention relates to electromagnetic devices and methods for therapeutically treating body tissue, and more particularly to a device and a method for enhancing and regenerating nerve fibers using electromagnetic field frequencies.

### BACKGROUND

Nerves can be easily damaged in a traumatic event due to their vulnerable positions in the body. If a nerve is damaged, it does have the ability to regenerate, if its soma and a small portion of the neurilemma remain. The nerve begins the process by destroying the nerve distal to the site of injury allowing Schwann cells, basal lamina, and the neurilemma near the injury to begin producing a regeneration tube. Nerve growth factors are produced causing many nerve sprouts to bud. When one of the growth processes finds the regeneration tube, it begins to grow rapidly towards its original destination guided the entire time by the regeneration tube. Nerve regeneration is very slow and can take up to several months to complete. While this process does repair some nerves, there will still be some functional deficit as the repairs are not perfect.

It is well known, according to Maguire E. A. et al (1997), that during the early stages of a recovery from a brain injury (e.g. accidental or stroke related) a physical stimulation is conventionally carried out in order to increase brain activity in those brain regions affected by the trauma. In many cases, there is a recognizable increase in brain activity in such brain regions or in close regions which restore activities that have been lost during the traumatic event. There are, however, nervous systems in the brain that cannot be stimulated by a physiotherapy, involving sensual activities such as vision, hear, smell or the like. Such nervous systems, once affected by trauma, will not be restored since there is no known way to artificially activate them.

In recent years it has been recognized that some brain systems operate in specific frequencies. For example, 10 Hz frequency is associated with the sympathetic nervous system that controls, among others, temporary changes in pupil size. Niehaus (et al. 2001) reported that stimulation of the brain in 10 hz frequency has an effect on the autonomic nervous system. It is shown to be possible to stimulate the sympathetic nervous system by artificial electromagnetic transmissions operating at 10 Hz, with no significant interference with other nerve pathways. Kahana (2006) showed that brain activity can be traced using EEG (Electroencephalography).

Several techniques aiming at nerve regeneration are published. U.S. Pat. No. 6,436,129 describes a method and apparatus for stimulating nerve regeneration by applying thermal energy to one or more nerve segments adjacent a damaged region of the nerve to promote rapid and more extensive growth of the nerve cells. The thermal energy is delivered through a probe configured to be used in proximity to the injured nerve, for example, in an open surgery procedure or arthroscopic or microscopic surgical environment.

Patent application WO2009003025 describes a grooved electrode being implanted inside a human body which when placed in the proximity of a neuron cell can guide the growth of said neuron cell and limit contacts of other neuron cells to said electrode.

Several patent applications describe photobiomodulation or photostimulation techniques for nerves stimulation. For example US patent application 20100016783 describes application of initial energy, i.e. laser therapy to a subject to produce an effect on a target structure directly or via a modulation agent.

It therefore remains a long felt and unmet need to provide novel means and methods for effectively and non-invasively regenerating specifically selected nerve fibers in a patient.

### SUMMARY OF THE INVENTION

It is thus an object of the present invention to disclose a method for regenerating nerve fibers of a subject. The method comprising the steps of: (a) selecting at least a target portion of the nervous system of a subject with a lesion in the nervous system; (b) detecting electromagnetic wave profile of said at least a target portion of said nervous system of said subject; (c) comparing wave profiles of said subject with wave profiles of normal patients; and (d) defining a treatment protocol; wherein said step of defining said treatment protocol includes selecting compensating electromagnetic waves adapted for correcting anomalous wave profiles associated with said nervous system lesions and transmitting to said subject said protocol of compensating electromagnetic waves to provide a resonance effect; thereby inducing regeneration of nerve fibers at said nervous system lesions and regions proximate to said lesions.

It is another object of the current invention to disclose a method as defined in any of the above, additionally comprising steps of selecting at least a target portion of the nervous system, wherein said subject target portion of the nervous system is not associated with a lesion, and said step of defining said treatment protocol includes selecting compensating electromagnetic waves adapted for correcting non lesion associated deficits in said subjects.

It is another object of the current invention to disclose a method as defined in any of the above, additionally comprising steps of transmitting a variety of electromagnetic waves of specified frequencies in a homogeneous field covering at least a portion of the volume of said at least a target portion of the nervous system of said subject which is resonated with said specified frequencies.

It is another object of the current invention to disclose a method as defined in any of the above, additionally comprising steps of transmitting electromagnetic field frequencies in the range of between about 0.01 Hz (DC) to about 100 Hz.

It is another object of the current invention to disclose a method as defined in any of the above, additionally comprising steps of transmitting electromagnetic field frequencies in intensity ranges from about 10̂-6 Gauss to about 100 Gauss.

It is another object of the current invention to disclose a method as defined in any of the above, additionally comprising steps of producing EMF generated by at least one oscillating wave.

It is another object of the current invention to disclose a method as defined in any of the above, additionally comprising steps of transmitting electromagnetic field frequencies characterized by pulse duration ranges from minutes to hours.

It is another object of the current invention to disclose a method as defined in any of the above, additionally comprising steps of providing at least one session of said treatment protocol at intervals selected from the group consisting of once a day, more than once per day, once per several days and any combination thereof.

It is another object of the current invention to disclose a method as defined in any of the above, additionally comprising steps of providing said at least one session of treatment protocol once a day for a period of days, weeks or months.

It is another object of the current invention to disclose a method as defined in any of the above, additionally comprising steps of enhancing nerve fiber regeneration by increasing at least one nerve fiber parameter selected from the group consisting of size, length, thickness and any combination thereof.

It is another object of the current invention to disclose a method as defined in any of the above, additionally comprising steps of inducing regeneration of damaged, injured or pathological, semi healthy or healthy nerve fibers or nerve fibers with a lesion or trauma.

It is another object of the current invention to disclose a method as defined in any of the above, additionally comprising steps of decreasing the symptoms of at least one disease or syndrome or condition selected from the group consisting of: nervous system lesions or damage, brain injuries (trauma), stroke, heart transplant, movement syndromes, digestion syndromes, breathing syndromes, heart syndromes, spinal cord syndromes, spinal disc herniation.

It is another object of the current invention to disclose a method as defined in any of the above, additionally comprising steps of defining said treatment protocol adapted for determining frequency, level and duration of the current supplied to said transmitter.

It is another object of the current invention to disclose a method as defined in any of the above, additionally comprising steps of defining said treatment protocol adapted for determining frequency, level, duration, periodicities and any combination thereof of said compensating electromagnetic waves.

It is another object of the current invention to disclose a method as defined in any of the above, additionally comprising steps of selecting said electromagnetic wave measuring device from the group consisting of: EEG, MEG, MRI, PET, any other acceptable monitoring or electromagnetic wave measuring means and any combination thereof.

It is another object of the current invention to disclose a method as defined in any of the above, additionally comprising steps of configuring said measuring device for evaluating the results of the electromagnetic waves transmission.

It is another object of the current invention to disclose a method as defined in any of the above, additionally comprising steps of evaluating said nerve fiber regeneration or neurogenesis by at least one parameter or test selected from the group consisting of relative leg placement test (FLP), relative front legs placement (FPS), relative neurological severity score (NSS), brain scans, nerve fiber scans, nerve fiber activity analysis, subventricular zone (SVZ) or subependymal zone or subgranular zone examination, MRI, PET, CT and any other available clinical test and any combination thereof.

It is another object of the current invention to disclose a method as defined in any of the above, additionally comprising steps of configuring said apparatus for decreasing the recovery period after nerve fiber damage or trauma.

It is another object of the current invention to disclose a method as defined in any of the above, additionally comprising steps of configuring said transmitter adapted for providing said electromagnetic wave useful for regenerating nerve fibers by means of inducing a resonance effect between the frequency profile of the transmitted electromagnetic wave and the profile of the natural tissue or region of interest of said subject.

It is another object of the current invention to disclose a method as defined in any of the above, additionally comprising steps of defining said resonance effect by the enhanced activity detected by nerve fiber analysis.

It is another object of the current invention to disclose a method as defined in any of the above, additionally comprising steps of transmitting said resonance effect thereby generating an electro-chemical transmission in the nerve cells and neuron of the selected nervous system portion.

It is another object of the current invention to disclose a method as defined in any of the above, additionally comprising steps of providing said CPU further including a database comprising data useful for profiling the electromagnetic wave of said at least a target portion of the nervous system of said subject in order to adjust a treatment protocol to said at least a target portion of the nervous system of said subject.

It is another object of the current invention to disclose a method as defined in any of the above, additionally comprising steps of configuring said CPU adapted to control said electromagnetic wave generator

It is another object of the current invention to disclose a method as defined in any of the above, additionally comprising steps of configuring said CPU adapted to control said treatment periodicities.

It is another object of the current invention to disclose a method as defined in any of the above, additionally comprising steps of defining said treatment protocol including a plurality of distinct frequencies within the range of the distinct tissue regions or target portions of the nervous system within said subject.

It is another object of the current invention to disclose a method as defined in any of the above, additionally comprising steps of configuring said transmitter comprising at least one coil for generating peripheral electromagnetic field.

It is another object of the current invention to disclose a method as defined in any of the above, additionally comprising steps of configuring said transmitter comprising at least one coil having a turn diameter sufficient to provide electromagnetic field to said target portion of the nervous system of said subject.

It is another object of the current invention to disclose a method as defined in any of the above, additionally comprising steps of configuring said transmitter comprising at least one coil providing a uniform field to said at least a target portion of the nervous system of said subject while said target portion is oriented in any convenient alignment.

It is another object of the current invention to disclose a method as defined in any of the above, additionally comprising steps of conforming said frequencies in accordance to specific tissues or body portions known to be capable of having an activity of interest.

It is another object of the current invention to disclose a method as defined in any of the above, additionally comprising steps of transmitting said electromagnetic field in at least one selected frequency for at least one session.

It is another object of the current invention to disclose a method as defined in any of the above, additionally comprising steps of synchronizing said transmission of said electromagnetic field with different nerve fiber types.

It is another object of the current invention to disclose a method as defined in any of the above, additionally comprising steps of regenerating nerve fibers so as to thickening and/or elongating of existing nerve fibers or bundles, generation of the new nerve fiber and any combination thereof.

It is another object of the current invention to disclose a computer readable medium comprising instructions for implementing a protocol useful for regenerating nerve fibers of a subject. The protocol comprising steps of: (a) detecting electromagnetic wave profile associated with a lesion of at least a target portion of said nervous system of said subject; (b) determining the electromagnetic wave characteristics of said lesion; and (c) defining a treatment regime; wherein said step of defining said treatment regime includes selecting compensating electromagnetic waves adapted for correcting anomalous wave profiles associated with said lesion; and transmitting to said subject said protocol of compensating electromagnetic waves to provide a resonance effect; thereby inducing regeneration of nerve fibers at said nervous system lesions and regions proximate to said lesions.

It is another object of the current invention to disclose a computer readable medium comprising instructions for implementing a protocol useful for regenerating nerve fibers as defined in any of the above, additionally comprising step of comparing wave profiles of said subject with wave profiles of normal patients.

It is another object of the current invention to disclose a computer readable medium comprising instructions for implementing a protocol useful for regenerating nerve fibers as defined in any of the above, additionally comprising step of detecting electromagnetic wave profile of at least a target portion of said nervous system of said subject, wherein said subject target portion of the nervous system is not associated with a lesion, and said step of defining said treatment protocol includes selecting compensating electromagnetic waves adapted for correcting non lesion associated deficits in said subjects.

It is another object of the current invention to disclose a computer readable medium comprising instructions for implementing a protocol useful for regenerating nerve fibers as defined in any of the above, additionally comprising step of transmitting a variety of electromagnetic waves of specified frequencies in a homogeneous field covering at least a portion of the volume of said at least a target portion of the nervous system of said subject which is resonated with said specified frequencies.

It is another object of the current invention to disclose a computer readable medium comprising instructions for implementing a protocol useful for regenerating nerve fibers as defined in any of the above, additionally comprising step of transmitting electromagnetic field frequencies in the range of between about 0.01 Hz (DC) to about 100 Hz.

It is another object of the current invention to disclose a computer readable medium comprising instructions for implementing a protocol useful for regenerating nerve fibers as defined in any of the above, additionally comprising step of transmitting electromagnetic field frequencies in intensity ranges from about 10̂-6 Gauss to about 100 Gauss.

It is another object of the current invention to disclose a computer readable medium comprising instructions for implementing a protocol useful for regenerating nerve fibers as defined in any of the above, additionally comprising step of producing EMF generated by at least one oscillating wave.

It is another object of the current invention to disclose a computer readable medium comprising instructions for implementing a protocol useful for regenerating nerve fibers as defined in any of the above, additionally comprising step of transmitting electromagnetic field frequencies characterized by pulse duration ranges from minutes to hours.

It is another object of the current invention to disclose a computer readable medium comprising instructions for implementing a protocol useful for regenerating nerve fibers as defined in any of the above, additionally comprising step of providing at least one session of said treatment protocol at intervals selected from the group consisting of once a day, more than once per day, once per several days and any combination thereof.

It is another object of the current invention to disclose a computer readable medium comprising instructions for implementing a protocol useful for regenerating nerve fibers as defined in any of the above, additionally comprising step of providing at least one session of said treatment protocol once a day for a period of days, weeks or months.

It is another object of the current invention to disclose a computer readable medium comprising instructions for implementing a protocol useful for regenerating nerve fibers as defined in any of the above, additionally comprising step of enhancing nerve fiber regeneration by increasing at least one nerve fiber parameter selected from the group consisting of size, length, thickness and any combination thereof.

It is another object of the current invention to disclose a computer readable medium comprising instructions for implementing a protocol useful for regenerating nerve fibers as defined in any of the above, additionally comprising step of inducing regeneration of damaged, injured or pathological, semi healthy or healthy nerve fibers or nerve fibers with a lesion or trauma.

It is another object of the current invention to disclose a computer readable medium comprising instructions for implementing a protocol useful for regenerating nerve fibers as defined in any of the above, additionally comprising step of decreasing the symptoms of at least one disease or syndrome or condition selected from the group consisting of: nervous system lesions or damage, brain injuries (trauma), stroke, heart transplant, movement syndromes, digestion syndromes, breathing syndromes, heart syndromes, spinal cord syndromes, spinal disc herniation.

It is another object of the current invention to disclose a computer readable medium comprising instructions for implementing a protocol useful for regenerating nerve fibers as defined in any of the above, additionally comprising step of defining said treatment protocol adapted for determining frequency, level and duration of the current supplied to said transmitter.

It is another object of the current invention to disclose a computer readable medium comprising instructions for implementing a protocol useful for regenerating nerve fibers as defined in any of the above, additionally comprising step of defining said treatment protocol adapted for determining frequency, level, duration, periodicities and any combination thereof of said compensating electromagnetic waves.

It is another object of the current invention to disclose a computer readable medium comprising instructions for implementing a protocol useful for regenerating nerve fibers as defined in any of the above, additionally comprising step of selecting said electromagnetic wave measuring device from the group consisting of: EEG, MEG, MRI, PET, any other acceptable monitoring or electromagnetic wave measuring means and any combination thereof.

It is another object of the current invention to disclose a computer readable medium comprising instructions for implementing a protocol useful for regenerating nerve fibers as defined in any of the above, additionally comprising step of configuring said measuring device for evaluating the results of the electromagnetic waves transmission.

It is another object of the current invention to disclose a computer readable medium comprising instructions for implementing a protocol useful for regenerating nerve fibers as defined in any of the above, additionally comprising step of evaluating said nerve fiber regeneration or neurogenesis by at least one parameter or test selected from the group consisting of relative leg placement test (FLP), relative front legs placement (FPS), relative neurological severity score (NSS), brain scans, nerve fiber scans, nerve fiber activity analysis, subventricular zone (SVZ) or subependymal zone or subgranular zone examination, MRI, PET, CT and any other available clinical test and any combination thereof.

It is another object of the current invention to disclose a computer readable medium comprising instructions for implementing a protocol useful for regenerating nerve fibers as defined in any of the above, additionally comprising step of configuring said apparatus for decreasing the recovery period after nerve fiber damage or trauma.

It is another object of the current invention to disclose a computer readable medium comprising instructions for implementing a protocol useful for regenerating nerve fibers as defined in any of the above, additionally comprising step of configuring said transmitter adapted for providing said electromagnetic wave useful for regenerating nerve fibers by means of inducing a resonance effect between the frequency profile of the transmitted electromagnetic wave and the profile of the natural tissue or region of interest of said subject.

It is another object of the current invention to disclose a computer readable medium comprising instructions for implementing a protocol useful for regenerating nerve fibers as defined in any of the above, additionally comprising step of defining said resonance effect by the enhanced activity detected by nerve fiber analysis.

It is another object of the current invention to disclose a computer readable medium comprising instructions for implementing a protocol useful for regenerating nerve fibers as defined in any of the above, additionally comprising step of transmitting said resonance effect thereby generating an electro-chemical transmission in the nerve cells and neuron of the selected nervous system portion.

It is another object of the current invention to disclose a computer readable medium comprising instructions for implementing a protocol useful for regenerating nerve fibers as defined in any of the above, additionally comprising step of providing said CPU further including a database comprising data useful for profiling the electromagnetic wave of said at least a target portion of the nervous system of said subject in order to adjust a treatment protocol to said at least a target portion of the nervous system of said subject.

It is another object of the current invention to disclose a computer readable medium comprising instructions for implementing a protocol useful for regenerating nerve fibers as defined in any of the above, additionally comprising step of configuring said CPU adapted to control said electromagnetic wave generator

It is another object of the current invention to disclose a computer readable medium comprising instructions for implementing a protocol useful for regenerating nerve fibers as defined in any of the above, additionally comprising step of configuring said CPU adapted to control said treatment periodicities.

It is another object of the current invention to disclose a computer readable medium comprising instructions for implementing a protocol useful for regenerating nerve fibers as defined in any of the above, additionally comprising step of defining said treatment protocol including a plurality of distinct frequencies within the range of the distinct tissue regions or target portions of the nervous system within said subject.

It is another object of the current invention to disclose a computer readable medium comprising instructions for implementing a protocol useful for regenerating nerve fibers as defined in any of the above, additionally comprising step of configuring said transmitter comprising at least one coil for generating peripheral electromagnetic field.

It is another object of the current invention to disclose a computer readable medium comprising instructions for implementing a protocol useful for regenerating nerve fibers as defined in any of the above, additionally comprising step of configuring said transmitter comprising at least one coil having a turn diameter sufficient to provide electromagnetic field to said target portion of the nervous system of said subject.

It is another object of the current invention to disclose a computer readable medium comprising instructions for implementing a protocol useful for regenerating nerve fibers as defined in any of the above, additionally comprising step of configuring said transmitter comprising at least one coil providing a uniform field to said at least a target portion of the nervous system of said subject while said target portion is oriented in any convenient alignment.

It is another object of the current invention to disclose a computer readable medium comprising instructions for implementing a protocol useful for regenerating nerve fibers as defined in any of the above, additionally comprising step of conforming said frequencies in accordance to specific tissues or body portions known to be capable of having an activity of interest.

It is another object of the current invention to disclose a computer readable medium comprising instructions for implementing a protocol useful for regenerating nerve fibers as defined in any of the above, additionally comprising step of transmitting said electromagnetic field in at least one selected frequency for at least one session.

It is another object of the current invention to disclose a computer readable medium comprising instructions for implementing a protocol useful for regenerating nerve fibers as defined in any of the above, additionally comprising step of synchronizing said transmission of said electromagnetic field with different nerve fiber types.

It is another object of the current invention to disclose a computer readable medium comprising instructions for implementing a protocol useful for regenerating nerve fibers as defined in any of the above, additionally comprising step of regenerating nerve fibers so as to thickening and/or elongating of existing nerve fibers or bundles, generation of the new nerve fiber and any combination thereof.

It is another object of the current invention to disclose an apparatus for regenerating nerve fibers in a subject, comprising: (a) at least one transmitter configured to generate an electromagnetic field (EMF) through at least a target portion of the nervous system of a subject; (b) at least one electromagnetic wave measuring device for detecting the electromagnetic wave profile of said at least a target portion of the nervous system of said subject; (c) at least one CPU for processing data concerned with detection of said electromagnetic wave profile of said at least a target portion of the nervous system of a subject, said CPU comprising a database for storing and analyzing wave profiles of normal patients and wave profiles of patients with nervous system lesions; and (d) at least one computer readable medium containing a predetermined protocol for operation of said transmitter. The at least one CPU has a first module for selection of compensating electromagnetic waves for correcting anomalous wave profiles of said patients with said nervous system lesions; and a second module for processing data concerned with transmission of said compensating electromagnetic waves to said nervous system lesions and regions proximate to said lesions.

It is another object of the current invention to disclose an apparatus as defined in any of the above, wherein said protocol comprises instructions to said transmitter for transmission of said compensating electromagnetic waves to provide a resonance effect; thereby inducing regeneration of nerve fibers at said nervous system lesions and regions proximate to said lesions.

It is another object of the current invention to disclose an apparatus as defined in any of the above, wherein said transmitter is adapted to produce EMF in predetermined frequency and intensity.

It is another object of the current invention to disclose an apparatus as defined in any of the above, configured to increase at least one nerve fiber parameter selected from the group consisting of size, length, thickness and any combination thereof.

It is another object of the current invention to disclose an apparatus as defined in any of the above, configured to induce regeneration of damaged, injured or pathological, semi healthy or healthy nerve fibers or nerve fibers with a lesion or trauma.

It is another object of the current invention to disclose an apparatus as defined in any of the above, wherein said transmitter produces EMF at frequency range of about 0.01 Hz (DC) to 100 Hz.

It is another object of the current invention to disclose an apparatus as defined in any of the above, wherein said transmitter produces EMF generated by at least one oscillating wave.

It is another object of the current invention to disclose an apparatus as defined in any of the above, wherein the electromagnetic wave is administered in the intensity of 10̂-6 to 100 Gauss.

It is another object of the current invention to disclose an apparatus as defined in any of the above, wherein the electromagnetic wave is administered in the duration of minutes to about an hour.

It is another object of the current invention to disclose an apparatus as defined in any of the above, wherein said apparatus is adapted to decrease the symptoms of at least one disease or syndrome or condition selected from the group consisting of: nervous system lesions or damage, brain injuries (trauma), stroke, heart transplant, movement syndromes, digestion syndromes, breathing syndromes, heart syndromes, spinal cord syndromes, spinal disc herniation.

It is another object of the current invention to disclose an apparatus as defined in any of the above, wherein said predetermined protocol is adapted for determining frequency, level and duration of the current supplied to said transmitter.

It is another object of the current invention to disclose an apparatus as defined in any of the above, wherein said predetermined protocol is adapted for determining frequency, level, duration, periodicities and any combination thereof of said electromagnetic waves.

It is another object of the current invention to disclose an apparatus as defined in any of the above, wherein said electromagnetic wave measuring device is selected from the group consisting of: EEG, MEG, MRI or any other acceptable monitoring or measuring means and any combination thereof.

It is another object of the current invention to disclose an apparatus as defined in any of the above, wherein said measuring device is configured for evaluating the results of the electromagnetic waves transmission.

It is another object of the current invention to disclose an apparatus as defined in any of the above, wherein said nerve fiber regeneration or neurogenesis is evaluated by at least one parameter, test or scanning or imaging device selected from the group consisting of relative leg placement test (FLP), relative front legs placement (FPS), relative neurological severity score (NSS), brain scans, nerve fiber scans, nerve fiber activity analysis, subventricular zone (SVZ) or subependymal zone or subgranular zone examination, MRI, PET, CT and any other available clinical test and any combination thereof.

It is another object of the current invention to disclose an apparatus as defined in any of the above, configured for decreasing the recovery period after nerve fiber damage or trauma.

It is another object of the current invention to disclose an apparatus as defined in any of the above, wherein said transmitter is adapted for providing said electromagnetic wave useful for regenerating nerve fibers by means of inducing a resonance effect between the frequency profile of the transmitted electromagnetic wave and the wave frequency profile of the natural tissue or region of interest of said subject.

It is another object of the current invention to disclose an apparatus as defined in any of the above, wherein said resonance effect is defined by the enhanced activity detected by nerve fiber analysis.

It is another object of the current invention to disclose an apparatus as defined in any of the above, wherein said resonance effect generates an electro-chemical transmission in the nerve cells and/or neuron of the determined nervous system.

It is another object of the current invention to disclose an apparatus as defined in any of the above, wherein said CPU further includes a database comprising data useful for profiling the electromagnetic wave of said at least a target portion of the nervous system of said subject in order to adjust a treatment protocol to said at least a target portion of the nervous system of said subject.

It is another object of the current invention to disclose an apparatus as defined in any of the above, wherein said CPU is further adapted to control said electromagnetic wave generator.

It is another object of the current invention to disclose an apparatus as defined in any of the above, wherein said CPU is further adapted to control the treatment periodicities.

It is another object of the current invention to disclose an apparatus as defined in any of the above, wherein said predetermined protocol includes a plurality of distinct frequencies within the range of the distinct tissue regions or target portions of the nervous system within said subject.

It is another object of the current invention to disclose an apparatus as defined in any of the above, wherein said transmitter comprises at least one coil for generating peripheral electromagnetic field.

It is another object of the current invention to disclose an apparatus as defined in any of the above, wherein said at least one coil is a Helmholtz coil, having a turn diameter sufficient to provide electromagnetic field to said target portion of the nervous system of said subject.

It is another object of the current invention to disclose an apparatus as defined in any of the above, wherein said apparatus comprises at least one coil providing a uniform field to said at least a target portion of the nervous system of said subject while said target portion is oriented in any convenient alignment.

It is another object of the current invention to disclose an apparatus as defined in any of the above, wherein said frequencies are conformed in accordance to specific tissues or body portions known to be capable of having an activity of interest.

It is another object of the current invention to disclose an apparatus as defined in any of the above, wherein said electromagnetic field is transmitted in at least one selected frequency for at least one session.

It is another object of the current invention to disclose an apparatus as defined in any of the above, wherein said transmission of said electromagnetic field is synchronized with different nerve fiber types.

It is another object of the current invention to disclose an apparatus as defined in any of the above, wherein said nerve regeneration procedure results in the thickening and/or elongation of existing nerve fibers or bundles, generation of the new nerve fibers and any combination thereof.

It is another object of the current invention to disclose a method for regenerating nerve fibers of a subject. The method comprising the steps of: (a) selecting at least a target portion of the nervous system of a subject with a lesion in the nervous system; (b) providing the apparatus as defined in any of the above; (c) detecting electromagnetic wave profile of said at least a target portion of said nervous system of said subject; (d) comparing wave profiles of said subject with wave profiles of normal patients; and (e) defining a treatment protocol; wherein said step of defining said treatment protocol includes selecting compensating electromagnetic waves adapted for correcting anomalous wave profiles associated with said nervous system lesions and transmitting to said subject said protocol of compensating electromagnetic waves to provide a resonance effect; thereby inducing regeneration of nerve fibers at said nervous system lesions and regions proximate to said lesions.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 presents a graphic representation of relative Forward Leg Position (FLP) or FPS measurements during stroke recovery, in accordance with an embodiment of the present invention;

FIG. 2 presents a graphic representation of relative neurological severity score (NSS) measurements during stroke recovery, in accordance with an embodiment of the present invention;

FIG. 3 presents MRI images of the subventricular zone (SVZ) during stroke recovery in treated rats as compared to control rats, in accordance with an embodiment of the present invention;

FIG. 4 presents images of nerve fiber regeneration of the corpus callosum after stroke procedure in treated rats as compared to control rats;

FIG. 5 presents images of nerve fiber regeneration of the Fronix-Fimbria after stroke procedure in treated rats as compared to control rats;

FIG. 6 presents images of the Corpus callosum demonstrating nerve fiber regeneration after stroke procedure in accordance with an embodiment of the present invention;

FIG. 7 presents images of the Internal capsule demonstrating nerve fiber regeneration after stroke procedure in accordance with an embodiment of the present invention;

FIG. 8 presents images of the Fornix fimbria demonstrating nerve fiber regeneration after stroke procedure in accordance with an embodiment of the present invention;

FIG. 9 presents images of the Anterior commissure demonstrating nerve fiber regeneration after stroke procedure in accordance with an embodiment of the present invention; and

FIG. 10 presents a schematic diagram of a method for regenerating nerve fibers of a subject, in accordance with an embodiment of the present invention.

### DETAILED DESCRIPTION

The following description is provided so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide device and method for stimulating the brain or different nervous system of a subject by means of the resonance effect between the frequency of the electromagnetic field directed to the subject and the natural wave frequency of at least a target portion of the nervous system of said subject.

The present invention provides a method for regenerating nerve fibers of a subject comprising the steps of: (a) selecting at least a target portion of the nervous system of a subject with a lesion in the nervous system; (b) detecting electromagnetic wave profile of said at least a target portion of said nervous system of said subject; (c) comparing wave profiles of said subject with wave profiles of normal patients; and (d) defining a treatment protocol; wherein said step of defining said treatment protocol includes selecting compensating electromagnetic waves adapted for correcting anomalous wave profiles associated with said nervous system lesions and transmitting to said subject said protocol of compensating electromagnetic waves to provide a resonance effect; thereby inducing regeneration of nerve fibers at said nervous system lesions and regions proximate to said lesions.

According to one embodiment, it is within the scope of the method disclosed above, wherein said subject target portion of the nervous system is not associated with a lesion, and said step of defining said treatment protocol includes selecting compensating electromagnetic waves adapted for correcting non lesion associated deficits in said subjects.

It is herein acknowledged that a nervous system functional unit comprises axons, neurons and synapses, and the transmission between them enables a particular function. The functional unit described herein operates as an ad hoc network of neuronal fibers connected in a pathway to achieve certain goals.

According to a core aspect, the method and device of the present invention increases the thickness, length and size of nerve fibers (both the injured fibers and the healthy ones), thus allowing better function even to existing systems.

It is further within the scope that the apparatus and method of the present invention not only treat brain related malfunctions, but also increase nerve fibers in other parts of the human body.

In a certain embodiment, the mode of action of the invention is by resonating at least a portion of the nervous system transmissions, i.e. nerve fibers with a predetermined selected frequency according to a predetermined protocol. Without being bounded by theory, the present invention works on the principle that in addition to characteristic frequencies of electrical impulses traveling through neural networks, and chemical pulses across synapses at predetermined wave frequencies, the present invention, for the first time, provides means of resonating the informational signals and frequencies at which these informational waves are propagated.

Certain aspects of the present invention can be understood by considering the example of a subject who has had a stroke, and has subsequently been noted to have a lesion in a specific portion of the nervous system. When electromagnetic (EM) waves of the correct frequency are transmitted to the desired portion of the nervous system, it will resonate as well as its surrounding regions, and nerve fibers in the lesion region will be regenerated, i.e. by thickening and/or elongating of existing nerve fibers or by generating new fibers, thus at least partially repairing the deficit.

Another example of usage of the device and method of the present invention is by considering a heart transplant, where regeneration of new nerve fibers leading to and from the transplant and connecting it to the central nervous system of the recipient body is a crucial process for a successful transplantation of the organ in the recipient body. This novel procedure will reduce the need for the conventionally used device that regulates heart beats.

According to some aspects of the invention, the effect obtained is an increased activity in the specified portion of the nervous system, which will induce regeneration of nerve fibers in neural networks having the pathology or lesion of interest, thus initiating new nerve fibers that will produce new pathways or will be engaged in the existing pathways at the nervous system region of interest.

According to a further aspect of the invention, the predetermined protocol is a treatment profile, comprising different frequencies, transmission sessions and intensities. The protocol is adjusted to at least a portion of the nervous system of interest of a specific subject.

The present invention further provides a device and method that by transmitting electromagnetic waves in a specific protocol, increase the size, length and/or thickness of the nerve fibers connecting to a specific organ or tissue and by that allowing it to function better, or in other words, resolve unstable operation by enhancing the nerves controlling the specific organ or tissue operation.

According to further aspects, the device and method of the present invention are configured to regenerate both injured fibers and healthy ones. It is thus within the scope that the present invention is configured to allow better function even to existing systems of the nervous system.

It is further within the scope of the invention that the apparatus and method disclosed herein are adapted to alleviate or improve, not only brain related malfunctions, but also to increase nerve fiber regeneration in other parts of the body. Research article Discrimination of Motor Imagery-Induced EEG Patterns in Patients with Complete Spinal Cord Injury, Pfurtscheller G et al. 2009 (Computational Intelligence and Neuroscience) is incorporated herein by reference, showing that different activities can be associated with different operation wavelengths. The present invention demonstrates the usage specific frequencies and intensities in a predetermined protocol to regenerate nerve fibers for a specific body function such as movement, digestion, breathing and the heart.

The device and method of the present invention are configured to increase the size, length and/or thickness of the nerve fibers connecting to the specific organ and by that allow it to function better, or in other words, resolve unstable operation by increasing the nerves controlling the organ operation. For example, in the case of a heart transplant, this may help to regenerate the nerve fibers to and from the new heart which will enable it to function highly better. A significant advantage of using the herein disclosed apparatus and method is reducing the need for using a transplanted or otherwise invasive device that regulates heart beats. In a further aspect, nerve systems that might suffer injury can be regenerated. These new nerve fibers will be able to reduce the impact of the injury, i.e. in the case of spinal injuries.

As used herein, the term ‘plurality’ refers in a non-limiting manner to any integer equal to or greater than 1.

The term ‘about’ refers herein to a value being ±25% of the defined measure.

The term ‘approximately’ refers herein a value being ±25% of the defined measure.

The term “nervous system” as used herein should be generally understood as including any nervous system or pathway within the brain and other peripheral systems. It mainly includes two main parts, the central nervous system (CNS) and the peripheral nervous system (PNS). The CNS contains the brain and spinal cord. The PNS consists mainly of nerves, which are long fibers that connect the CNS to other parts of the body. The PNS mainly includes motor neurons, mediating voluntary movement, the autonomic nervous system, comprising the sympathetic nervous system and the parasympathetic nervous system and regulating involuntary functions, and the enteric nervous system, whose function is to control the gastrointestinal system. At the cellular level, the nervous system is defined by the presence of a special type of cell, called the neuron, also known as a “nerve cell”.

The term “nerve fiber” or “nerve fibers” refers herein, in a non limiting manner to a threadlike extension of a nerve cell or neuron in the nervous system, especially a prolonged axon that conducts nerve impulses. A nerve fiber also refers to an axon or dendrite of a neuron. The nerve fiber consists of an axon and optionally, myelin sheath. There are nerve fibers in the central nervous system and peripheral nervous system. A nerve fiber may be myelinated and/or unmyelinated. In the central nervous system (CNS), myelin is produced by oligodendroglia cells. Schwann cells form myelin in the peripheral nervous system (PNS). In the PNS, Schwann cells can also produce a covering for an axon which does not consist of myelin. Specifically, a peripheral nerve fiber consists of an axon, myelin sheath, Schwann cells and its endoneurium. It is noted that in the central nervous system there are no endoneurium and Schwann cells. Components of peripheral nerve fiber include: an axon (or a long dendrite of sensory fiber), axolemma, optionally myelin sheath, Schwann's sheath (neurolemma) and endoneurium.

According to further aspects, in the central nervous system, nerve fibers differ in terms of size, conduction velocity, and presence or lack of myelin. In a further aspect, a bundle of nerve fibers constitutes a tract in the central nervous system. The pyramidal tract and extrapyramidal tracts have long nerve fibers that descend from the brain to the spinal cord. These fibers have an important role in motor control, and are known as descending tracts. There are other bundles of nerve fibers in the CNS that are referred to as ascending tracts. These tracts carry information (i.e. sensory information) from the periphery to the different areas of the brain (such as the cerebral cortex, cerebellum, and brain stem).

It is herein acknowledged that nerve fibers can be classified in several ways. For example, fibers of the upper motor neurons, including;
pyramidal system: Corticospinal tract (or Pyramidal tract) and Corticobulbar tract;
Extrapyramidal tracts: Vestibulospinal tract, Reticulospinal tract, Tectospinal tract, Rubrospinal tract, Olivospinal tract;
Descending autonomic fibers: Hypothalamobulbar fibers and Hypothalamospinal fibers;

Ascending nerve fibers, including;
Spinothalamic tract: Anterior spinothalamic tract and Lateral spinothalamic tract;
Fasciculus cuneatus
Fasciculus gracilis
Spinoreticular tract
Spinotectal tract
Spinocerebellar tract, including: Posterior spinocerebellar tract and Anterior spinocerebellar tract; Cuneocerebellar tract and Rostral spinocerebellar tract;
Spinocervical tract

Peripheral nerve fiber types, including;
Sensory nerve fibers (afferent fibers)
Motor nerve fibers (efferent fibers)
Autonomic nerve fibers (autonomic fibers)
sensory-motor (mixed nerve fiber)

Peripheral nerve fibers can be classified based on their diameter:
Fibers having a large diameter and high conduction velocity, and are myelinated fibers (A group), including A alpha fibers (afferent or efferent fibers), A beta fibers (afferent or efferent fibers), A gamma fibers (efferent fibers) and A delta fibers (afferent fibers);
Myelinated nerve fibers with a small diameter (B group). Generally, they are the preganglionic fibers of the autonomic nervous system and have a low conduction velocity.
Unmyelinated fibers having a small diameter and low conduction velocity (C group). These fibers include: Postganglionic fibers in the autonomic nervous system (ANS), Nerve fibers at the dorsal roots (IV fiber); These fibers may carry the following sensory information: nociception (pain), temperature, touch, pressure and itch.

The term “neural network” used herein refers to a network or circuit of biological neurons. The term further refers to artificial neural networks, which comprises artificial neurons or nodes. The neural network has distinct usages such as biological neural network. Biological neuronal networks are connected or functionally related to the nervous system. In the field of neuroscience, they are often identified as groups of neurons that perform a specific physiological function in laboratory analysis.

A biological neural network is composed of a group or groups of chemically connected or functionally associated neurons. All neurons are electrically excitable, maintaining voltage gradients across their membranes by means of metabolically driven ion pumps, which combine with ion channels embedded in the membrane to generate intracellular-versus-extracellular concentration differences of ions such as sodium, potassium, chloride, and calcium. Changes in the cross-membrane voltage can alter the function of voltage-dependent ion channels. If the voltage changes by a large enough amount, an all-or-none electrochemical pulse called an action potential is generated, which travels rapidly along the cell's axon, and activates synaptic connections with other cells when it arrives. A single neuron may be connected to many other neurons and the total number of neurons and connections in a network may be extensive. The connections, known as synapses, are formed from axons to dendrites, though dendrodendritic microcircuits and other connections are possible. An axon, also known as a nerve fiber, is a long, slender projection of a nerve cell, or neuron that typically conducts electrical impulses away from the neuron's cell body. Axons are in effect the primary transmission lines of the nervous system, and as bundles they help make up nerves. Axons make contact with other cells at junctions called synapses. Synapses are essential to neuronal function. Neurons are cells that are specialized to pass signals to individual target cells. The key to neural function is the synaptic signaling process. At a synapse, the membrane of the axon closely adjoins the membrane of the target cell, and special molecular structures serve to transmit electrical or electrochemical signals across the gap. Some synaptic junctions appear partway along an axon as it extends called en passant (“in passing”) synapses. Other synapses appear as terminals at the ends of axonal branches. A single axon can innervate multiple parts of the brain and generate thousands of synaptic terminals.

Apart from the electrical signaling, there are other forms of signaling that arise from neurotransmitter diffusion.

The term “nerve fiber regeneration” or “nerve regeneration” used herein is also known as neuroregeneration and generally refers to the regrowth or repair of nervous tissues, cells or cell products. Such mechanisms may include generation of new neurons, glia, axons, myelin, or synapses. Neuroregeneration differs between the peripheral nervous system (PNS) and the central nervous system (CNS) by the functional mechanisms and especially the extent and speed. When an axon is damaged, the distal segment undergoes Wallerian degeneration, losing its myelin sheath. The proximal segment can either die by apoptosis or undergo the chromatolytic reaction, which is an attempt at repair. In the CNS, synaptic stripping occurs as glia foot processes invade the dead synapse. The present invention provides novel and unexpected means and methods for rapidly and effectively regenerating nerve fibers of healthy and injured tissues within the nervous system.

The term “electromagnetic field” or “EMF” or “EM” refers to a physical field produced by electrically charged objects. It affects the behavior of charged objects in the vicinity of the field. The electromagnetic field extends indefinitely throughout space and describes the electromagnetic interaction. The field can be viewed as the combination of an electric field and a magnetic field. The electric field is produced by stationary charges, and the magnetic field by moving charges (currents); these two are often described as the sources of the field. The way in which charges and currents interact with the electromagnetic field is described by Maxwell's equations and the Lorentz force law.

The electromagnetic field can be regarded as a continuous field, propagated in a wavelike manner. Electromagnetic waves are created by the vibration of an electric charge. This vibration creates a wave which has both an electric and a magnetic component. The electromagnetic spectrum is the range of all possible frequencies of electromagnetic radiation. The electromagnetic spectrum extends from below the low frequencies used for radio communication to gamma radiation at the short-wavelength (high-frequency) end. Electromagnetic waves are typically described by any of the following three physical properties: the frequency f, wavelength λ, or photon energy E.

The types of electromagnetic radiation are broadly classified into the following classes: Gamma radiation, X-ray radiation, ultraviolet radiation, visible radiation, infrared radiation, terahertz radiation, microwave radiation and radio waves. The apparatus of the present invention is configured to provide EMF at the frequencies of about 0.01 (DC) to about 100 Hz, and in the power of about 10̂-6 to 100 Gauss.

The term “scanning or imaging device” or “measuring device” used herein refers to any measurement and/or recording techniques designed to produce images, such as electroencephalography (EEG), magnetoencephalography (MEG), electrocardiography (EKG), and others, which produce data susceptible to be represented as maps forms of medical imaging, radiographic diagnostic techniques, imaging modalities such as PET and magnetic resonance imaging instrument (MRI scanner), Positron emission tomography (PET), Computed tomography (CT).

By generating an electromagnetic field having the same frequency as the determined nervous system, a resonance effect is created which generates electro-chemical transmission in the nerve cells and neuron of the determined nervous region, therefore promoting the process of regenerating brain cells.

The device of the present invention relays on the fact that each region in the nervous system has its own operating frequency. This means that the transmission of electromagnetic waves do not have to be targeted to a specific region of the brain. Instead, the transmission of a particular frequency over a relevant portion of the nervous system will exert an effect. Therefore parts of the nervous system, having different operating frequencies, will not resonate and mainly a region of interest will be activated in response to the transmission.

The present invention further demonstrates that the nervous system frequency is one of the factors governing the activation of the nervous system.

The present invention further presents that different parts of the nervous system are activated by the transmission of different field intensities. The examples further demonstrates that frequency, intensity and duration of the treatment has different outcomes, such that transmission is a 3D array when frequency, intensity and duration play a role in determining the exact nervous system and its desired section is targeted for enhancing its operational capabilities, thus reducing the symptoms of the patient syndrome or condition.

The term “frequency” as used herein relates to a direct transmission in a given frequency or transmission of at least two frequencies generated by one oscillating wave or by a combination of two or more waves which results in transmission of having the given frequencies or wavelengths.

The term “resonance” or “resonance effect” as used herein relates to the effect that occurs when a given electromagnetic field transmission, having the similar frequency of the natural operating frequency of a given nervous system, generates a neurotically activity or neurogenesis effect in that system. Each portion of the nervous system can be described as a set of electro-chemical networks which has a specified operating frequency which allows it to transmit information from one nerve cell to other nerve cells of the same portion or functional part of the nervous system, i.e. at the same time. It is within the scope of the present invention that different operating frequencies separate the nervous systems functionality allowing the brain or other part of the nervous system (such as the peripheral nervous system) to function in parallel without inter-nervous system interferences. When the transmitter is turned on, it endues transmission within the specified portion of the nervous system having the same frequency and by that enhances its operation. Once the transmission ends, the nervous system regains its normal activity.

The present invention provides a method for regenerating nerve fibers of a subject comprising the steps of: (a) selecting at least a target portion of the nervous system of a subject with a lesion in the nervous system; (b) detecting electromagnetic wave profile of said at least a target portion of said nervous system of said subject; (c) comparing wave profiles of said subject with wave profiles of normal patients; and (d) defining a treatment protocol; wherein said step of defining said treatment protocol includes selecting compensating electromagnetic waves adapted for correcting anomalous wave profiles associated with said nervous system lesions and transmitting to said subject said protocol of compensating electromagnetic waves to provide a resonance effect; thereby inducing regeneration of nerve fibers at said nervous system lesions and regions proximate to said lesions.

It is within the scope of the method disclosed above, wherein said subject target portion of the nervous system is not associated with a lesion, and said step of defining said treatment protocol includes selecting compensating electromagnetic waves adapted for correcting non lesion associated deficits in said subjects.

The present invention further provides an apparatus for regenerating nerve fibers in a subject and method thereof. The apparatus comprising: (a) at least one transmitter configured to generate an electromagnetic field (EMF) through at least a target portion of the nervous system of a subject; (b) at least one electromagnetic wave measuring device for detecting the electromagnetic wave profile of said at least a target portion of the nervous system of said subject; (c) at least one CPU for processing data concerned with detection of said electromagnetic wave profile of said at least a target portion of the nervous system of a subject, said CPU comprising a database for storing and analyzing wave profiles of normal patients and wave profiles of patients with nervous system lesions; and (d) at least one computer readable medium containing a predetermined protocol for operation of said transmitter. In this way, the at least one CPU has a first module for selection of compensating electromagnetic waves for correcting anomalous wave profiles of said patients with said nervous system lesions; and a second module for processing data concerned with transmission of said compensating electromagnetic waves to said nervous system lesions and regions proximate to said lesions.

It is within the scope of the invention that the protocol as defined above comprises instructions to the transmitter for transmission of the compensating electromagnetic waves to provide a resonance effect; thereby inducing regeneration of nerve fibers at the nervous system lesions and regions proximate to the lesions.

The present invention demonstrates that the innovative technology can identify the waves frequencies which can be associated to a specific disorder of the nervous system, and the appropriate waves are utilized as compensating EM waves in the recovery treatment as determined i.e. according to the MEG (magnetoencephalography) data. This is performed by subjecting a patient to an ongoing series of electromagnetic exposures and supplementary treatment, and then targeting the nervous system which is with the need to be enhanced or treated. The rehabilitation process may be accompanied by para-medical treatment that will enhance the effect of the device and will relate to other aspects of recovery which are not necessary related directly to medical issues.

According to a further embodiment, an optional treatment procedure includes measuring electromagnetic wave profile by a measuring device such as the MEG, or identifying anomalous wave profile, for selecting which nervous system will be addressed in the recovery treatment. The patient is subjected to an ongoing series of electromagnetic exposures and supplementary treatment targeting the nervous system which is specified to be enhanced.

According to a further embodiment, the apparatus comprises a measuring device such as MEG for detecting subject selected nervous system wave frequency, at least one transmitter comprising at least one coil, configured to generate an electromagnetic field through the specific nervous system portion of a subject, at least one CPU for processing and analyzing data concerned with detection of wave frequency of a subject. The CPU comprising a database for storing and analyzing wave profiles of normal patients and wave profiles of patients with nervous system lesions. The apparatus further comprises at least one computer readable medium containing a predetermined protocol for transmission of the electromagnetic wave frequency profiles by the transmitter.

The at least one CPU comprises a database including the treatment procedures and the treatment results and controls the wave generator and the treatment timing.

In one embodiment, the at least one CPU has a first module for selection of compensating electromagnetic waves for correcting anomalous wave profiles of the patients with the nervous system lesions; and a second module for processing data concerned with transmission of said compensating electromagnetic waves to the nervous system lesions and regions proximate to said lesions.

According to a further embodiment, the protocol comprises instructions to the transmitter for transmission of the compensating electromagnetic waves to provide a resonance effect; thereby inducing regeneration of nerve fibers at the nervous system lesions and regions proximate to the lesions.

In contrast to the artificial stimulation using transcranial magnetic stimulation (TMS) that transmit direct energy to the targeted area in the brain assuming to generate natural pulses in the desired nervous system, the present innovation is configured to encourage transmission within the nervous system, by resonance effects in specific portions of the nervous system by transmitting electromagnetic field of low frequency waves ranging from 0.01 Hz (DC) to about 100 Hz. In one embodiment the exact frequency will be correlated to a nervous system portion of interest by a predetermined electromagnetic frequency profile.

In addition, it is herein demonstrated that the nervous system activity is not determined by frequency alone. The results presented show that treatment intensity (power and duration) has different effect on different parts of a given nervous system part or portion. It is further within the scope that parts of the nervous system are activated in different operating intensity, thus separating activity according to stimulation (natural or artificial). The range of intensity varies from 10̂-6 Gauss to 100 Gauss, and treatment time from minutes to hours.

The invention further relates to an apparatus comprising at least one transmitter configured to generate electromagnetic field, the transmitter comprises a positioning system configured to maintain the subject organ or body part in a predetermined position respective to at least one electrical coil. The coil provides electromagnetic field through the subject selected body portion, wherein the coil is electrically connected or connectable to an alternating current supply unit. The current supply unit is under control of a treatment profile processing unit capable of determining the frequency, the level or intensity and the duration of the current supplied to the at least one coil according to a nervous system of interest to be stimulated. It is noted that while the electromagnetic field is a peripheral one, and the specificity is established by choosing the exact frequency of the desired nervous fibers, there is no need to use a specific device for every distinct organ. On the contrary, while, for example, a limb nervous extend from the muscles of the limb through the spine up to the controlling region in the brain, the device of the present invention is designed to be sufficiently large or adjustable, to contain the entire body so as to be able to activate the nerve fiber thought its entire length. In this mode of action, the recovery process will be carried out from where it is most healthy to the parts where it is damaged.

The stimulation apparatus of the present invention preliminary activates the nervous system for increasing the nerve fiber regeneration and for finally enlarging the number of nerve fibers in the desired nervous system paths.

The invention further relates to a method of transmitting different electromagnetic fields of specified frequencies in a homogeneous field covering the entire nerve fiber length from the brain to the target tissue or organ which resonates with the specified nervous system. In one embodiment, the electromagnetic field frequencies varies from 0.01 Hz (DC) to 100 Hz, in intensities ranges from 10̂-6 Gauss to 100 Gauss and of transmitting duration ranges from minutes to hours.

The method further comprises steps of selecting a frequency within the range of frequencies for specific neural network known to be capable of having the activity of interest.

The method is performed by exposing the subject nervous system portion to a magnetic field and alternating the selected frequency, thereby directing the electromagnetic field toward a tissue of interest by means of resonance having the activity of interest.

The transmission of the electromagnetic field in the selected frequency may be repeated according to a predetermined protocol. Each session is for a predetermined duration during a time gap, within which the newly formed nerve fibers is initiated by synchronizing the transmission of the electromagnetic field with the cell cycle of nerve regeneration for furthermore stimulating the creation of the new nervous system paths of interest.

An additional aspect of the invention relates to a method for improving or enhancing the capabilities of a human nervous system, the method comprises subjecting at least a portion of the nervous system to a transmission of electromagnetic waves in both frequency and intensity similar to these expected from natural waves normally originating in paths of nervous system of interest, thereby selectively inducing nervous system paths of interest and increasing their activity.

In a preferred embodiment of the invention the transmission is repeated according to a predetermined protocol, each time for a predetermined duration. The protocol may rely on experience, experimentally obtained data tables and/or response curves, calculations, imaging of the specific nervous system, undergoing the protocol, thereby synchronizing the transmission with the specific nervous system natural electromagnetic field profile to generate a resonance effect to achieve regeneration of the desired nerve fibers.

The attribution of the EMF waves to a specific region of the nervous system is identified when they are in abnormal intensity and frequency profile. Compensatory EMF waves are generated artificially and directed to regions which are suspected of having a lesion or deficit. Transmitting the frequency and intensity of the specific compensatory electromagnetic waves encourages nerve fiber regeneration in the at least one nervous system portion of deficit or lesion.

Another embodiment of the present invention discloses an apparatus comprising: a nerve fiber wave measuring device, and a nerve fiber EMF wave transmission device.

The measuring device measures the nervous system frequency profile and further determines the nervous system region of those specific frequencies (preferably a MEG system) that are in subnormal intensity. The transmitting device is adapted to transmitting the frequency needed to invoke the regeneration treatment process. The present invention may further include physio-therapy, psychologists or other therapies to enhance the effect of the device and accelerate the “healing” process.

The nervous system waves measuring device is monitored by means of EEG, MEG or by any other acceptable monitoring means. The waves measuring device and the wave transmission device are associated to at least a selected nervous system portion and/or function.

In an alternative embodiment, the two devices may be integrated into one unit comprising two modules: a measuring module and a transmission module. The measuring module may comprise a sensing system (e.g. EEG or MEG or MRI or PET or any combination thereof) configured to define the characteristics of the elected nerve fiber waves which are related to the nervous system portion aimed to be stimulated. The sensing system further comprises analysis software or imaging algorithms configured to generate a correlation between the nervous system functional part and respective wave frequencies profile.

It is within the scope of the invention, wherein the same measuring system may be used for evaluating the results of the transmission sessions. For instance, a person intended to undergo the procedure will be exposed to specific transmission protocol which is associated with a specific nervous system lesion or function of interest. The increased activity of the waves of a specific frequency in the induced nervous system portion will be then monitored.

The EMF wave measuring device according the present invention comprises electromagnetic wave monitor comprising a MEG system, in a communication with the apparatus of the present invention. In a further embodiment, the wave measuring device may have visualization capabilities such as functional MRI and/or a brain positron emission tomography (PET), capable of detecting or imaging the nervous system activity in specific tissues or organs, therefore may be used in combination with the system and method of the present invention.

According to the present invention, subjecting the at least a portion of the nervous system to the electromagnetic oscillating field is carried out at frequencies similar or identical, to the frequencies detected in the measuring device and associated with the desired nervous fibers of interest.

It is herein emphasized that while the transmitted electromagnetic field is a peripheral one, and the focusing or specificity is done by selecting the exact frequency or frequency range of the desired nervous fibers. Thus, one advance of the current invention is that there is no need to generate a specific device for every distinct organ.

The electromagnetic oscillating field is generated by at least one coil or a set of coils while at least one coil is configured to generate electromagnetic fields resonating with the nervous system portion of interest, for enhancing its activity or nerve fiber regeneration.

According to a preferred embodiment of the invention, at least one coil, while two coils system might be applied by, such as a Helmholtz coil, having a turn diameter sufficient to contain at least a predetermined portion of the body, preferably the entire body. For example, while a limb nervous extend from the muscles of the limb through the spine up to the controlling region in the brain, the disclosed device is large enough to contain the entire body so it will be able to activate the nerve fiber thought its entire length, so it will be able to perform the recovery process from the most healthy part of the nerve fibers to the parts where it is damaged.

In certain aspects of the invention, in order to generate a homogeneous electromagnetic field in the volume between the coils, the diameter of the coil may be twice the distance between two similar portions thereof constituting a Helmholtz coil having a gap in between for receiving the body part of a treated person. If for example, the gap between the coil portions is 25 cm (10″), the diameter of the coil turns will be 50 cm (20″).

It is further within the scope, wherein the electromagnetic field induces the desired nervous system area by means of resonance effect between the electromagnetic field frequency and the natural wave frequency of the respective nerve fibers.

The number of coil turns is a matter of design. The coil turns and characteristics may depend on the current to be used for generating the electromagnetic field and/or on the thickness and resistance of the wire from which the coil is prepared. The coils within the apparatus are adapted to cover the desired treated volume in any convenient orientation, without the need to specifically locating it.

The present invention further comprises an alternating current supply unit configured to supply current to at least one coil, in a frequency, current level, and intensity and for time duration which can be determined according to predetermined treatment considerations.

Preferably, the apparatus of the present invention comprises a computerized processing unit or/and computer readable medium (e.g. a CPU), comprising a database with a treatment protocol which is selected from a plurality of predetermined treatment profiles stored in a memory unit either locally associated with the apparatus or available from a remote server.

The treatment profile is selected according to treated subject details; such details may include e.g. the patient's nervous system injury or lesion, location of the lesion or injured tissue, and a missing and/or reduced function of wave frequency and/or activity signals.

The CPU unit may be instructed to automatically control the current supply for supplying currents and intensities to the at least one coils, according to the selected protocol.

Since minor differences between the natural frequency of a nervous system portion of interest may occur between different people, and in order to allow usage for treatment of the CPU database, without preceding a specific analysis of a subject nervous system, the computerized system may be configured to work in a sweep or adjustable mode in which the frequency of the current is controlled during a treatment session. The database is further adapted for mapping predetermined nerve fibers activity.

According to a further embodiment, the apparatus and method are applied in order to regenerate nerve fibers associated with the activity and function of elected tissues or organs of the body, life expectancy and life quality of a subject. More specifically, the apparatus and method are applied for treating or improving the following syndromes or conditions: nervous system lesions or damage, brain injuries (trauma), stroke, heart transplant, movement syndromes, digestion syndromes, breathing syndromes, heart syndromes, spinal cord syndromes, spinal disc herniation.

It is within the scope of the invention to provide a method for regenerating nerve fibers of a subject comprising the steps of: (a) selecting at least a target portion of the nervous system of a subject with a lesion in the nervous system; (b) detecting electromagnetic wave profile of said at least a target portion of said nervous system of said subject; (c) comparing wave profiles of said subject with wave profiles of normal patients; and (d) defining a treatment protocol; wherein said step of defining said treatment protocol includes selecting compensating electromagnetic waves adapted for correcting anomalous wave profiles associated with said nervous system lesions and transmitting to said subject said protocol of compensating electromagnetic waves to provide a resonance effect; thereby inducing regeneration of nerve fibers at said nervous system lesions and regions proximate to said lesions.

It is further within the scope of the invention to disclose the method as defined in any of the above, wherein said subject target portion of the nervous system is not associated with a lesion, and said step of defining said treatment protocol includes selecting compensating electromagnetic waves adapted for correcting non lesion associated deficits in said subjects.

It is further within the scope to provide the method as defined in any of the above, comprising additional steps of transmitting a variety of electromagnetic waves of specified frequencies in a homogeneous field covering at least a portion of the volume of the at least a target portion of the nervous system of the subject which is resonated with the specified frequencies.

It is further within the scope to provide the method as defined in any of the above, The method according to claim 31, comprising an additional step of transmitting electromagnetic field frequencies in the range of between about 0.01 Hz (DC) to about 100 Hz.

It is further within the scope to provide the method as defined in any of the above, comprising an additional step of transmitting electromagnetic field frequencies in intensity ranges from about 10̂-6 Gauss to about 100 Gauss.

It is further within the scope to provide the method as defined in any of the above, comprising an additional step of producing EMF generated by at least one oscillating wave.

It is further within the scope to provide the method as defined in any of the above, comprising an additional step of transmitting electromagnetic field frequencies characterized by pulse duration ranges from minutes to hours.

It is further within the scope to provide the method as defined in any of the above, comprising an additional step of providing the treatment protocol that may vary from once per few days to several times a day. More particularly, the method as defined in any of the above may include a step of providing at least one session of said treatment protocol at intervals selected from the group consisting of once a day, more than once per day, once per several days and any combination thereof

It is further within the scope to provide the method as defined in any of the above, comprising an additional step of providing the treatment protocol once a day for a period of days, weeks or months.

It is further within the scope to provide the method as defined in any of the above, comprising an additional step of enhancing nerve fiber regeneration by increasing at least one nerve fiber parameter selected from the group consisting of size, length, thickness and any combination thereof.

It is further within the scope to provide the method as defined in any of the above, comprising an additional step of inducing regeneration of damaged, injured or pathological, semi healthy or healthy nerve fibers or nerve fibers with a lesion or trauma.

It is further within the scope to provide the method as defined in any of the above, comprising an additional step of decreasing the symptoms of at least one disease or syndrome or condition selected from the group consisting of: nervous system lesions or damage, brain injuries (trauma), stroke, heart transplant, movement syndromes, digestion syndromes, breathing syndromes, heart syndromes, spinal cord syndromes, spinal disc herniation.

It is further within the scope to provide the method as defined in any of the above, comprising an additional step of defining the treatment protocol adapted for determining frequency, level and duration of the current supplied to the transmitter.

It is further within the scope to provide the method as defined in any of the above, comprising an additional step of defining the treatment protocol adapted for determining frequency, level, duration, periodicities and any combination thereof of the compensating electromagnetic waves.

It is further within the scope to provide the method as defined in any of the above, comprising an additional step of selecting the electromagnetic wave measuring device from the group consisting of: EEG, MEG, MRI, PET, any other acceptable monitoring or electromagnetic wave measuring means and any combination thereof.

It is further within the scope to provide the method as defined in any of the above, comprising an additional step of configuring the measuring device for evaluating the results of the electromagnetic waves transmission.

It is further within the scope to provide the method as defined in any of the above, comprising an additional step of evaluating the nerve fiber regeneration or neurogenesis by at least one parameter or test selected from the group consisting of relative leg placement test (FLP), relative front legs placement (FPS), relative neurological severity score (NSS), brain scans, nerve fiber scans, nerve fiber activity analysis, subventricular zone (SVZ) or subependymal zone or subgranular zone examination, MRI, PET, CT and any other available clinical test and any combination thereof.

It is further within the scope to provide the method as defined in any of the above, comprising an additional step of configuring the apparatus for decreasing the recovery period after nerve fiber damage or trauma.

It is further within the scope to provide the method as defined in any of the above, comprising an additional step of configuring the transmitter adapted for providing the electromagnetic wave useful for regenerating nerve fibers by means of inducing a resonance effect between the frequency profile of the transmitted electromagnetic wave and the profile of the natural tissue or region of interest of the subject.

It is further within the scope to provide the method as defined in any of the above, comprising an additional step of defining the resonance effect by the enhanced activity detected by nerve fiber analysis.

It is further within the scope to provide the method as defined in any of the above, comprising an additional step of transmitting the resonance effect thereby generating an electro-chemical transmission in the nerve cells and neuron of the selected nervous system portion.

It is further within the scope to provide the method as defined in any of the above, comprising an additional step of providing the CPU further including a database comprising data useful for profiling the electromagnetic wave of the at least a target portion of the nervous system of the subject in order to adjust a treatment protocol to the at least a target portion of the nervous system of the subject.

It is further within the scope to provide the method as defined in any of the above, comprising an additional step of configuring the CPU adapted to control the electromagnetic wave generator

It is further within the scope to provide the method as defined in any of the above, comprising an additional step of configuring the CPU adapted to control the treatment periodicities.

It is further within the scope to provide the method as defined in any of the above, comprising an additional step of defining the treatment protocol including a plurality of distinct frequencies within the range of the distinct tissue regions or target portions of the nervous system within the subject.

It is further within the scope to provide the method as defined in any of the above, comprising an additional step of configuring the transmitter comprising at least one coil for generating peripheral electromagnetic field.

It is further within the scope to provide the method as defined in any of the above, comprising an additional step of configuring the transmitter comprising at least one coil having a turn diameter sufficient to provide electromagnetic field to the target portion of the nervous system of the subject.

It is further within the scope to provide the method as defined in any of the above, comprising an additional step of configuring the transmitter comprising at least one coil providing a uniform field to the at least a target portion of the nervous system of the subject while the target portion is oriented in any convenient alignment.

It is further within the scope to provide the method as defined in any of the above, comprising an additional step of conforming the frequencies in accordance to specific tissues or body portions known to be capable of having an activity of interest.

It is further within the scope to provide the method as defined in any of the above, comprising an additional step of transmitting the electromagnetic field in at least one selected frequency for at least one session.

It is further within the scope to provide the method as defined in any of the above, comprising an additional step of synchronizing the transmission of the electromagnetic field with different nerve fiber types.

It is further within the scope to provide the method as defined in any of the above, comprising an additional step of regenerating nerve fibers so as to thickening and/or elongating of existing nerve fibers or bundles, generation of the new nerve fiber and any combination thereof.

It is further within the scope to provide a computer readable medium comprising instructions for implementing a protocol useful for regenerating nerve fibers of a subject, said protocol comprising steps of: (a) detecting electromagnetic wave profile associated with a lesion of at least a target portion of said nervous system of said subject; (b) determining the electromagnetic wave characteristics of said lesion; and (c) defining a treatment regime. In this way the step of defining said treatment regime includes selecting compensating electromagnetic waves adapted for correcting anomalous wave profiles associated with said lesion; and transmitting to said subject said protocol of compensating electromagnetic waves to provide a resonance effect; thereby inducing regeneration of nerve fibers at said nervous system lesions and regions proximate to said lesions.

It is further within the scope to provide a computer readable medium comprising instructions for implementing a protocol as defined in any of the above, comprising additional step of comparing wave profiles of said subject with wave profiles of normal patients.

It is further within the scope to provide a computer readable medium comprising instructions for implementing a protocol as defined in any of the above, wherein said subject target portion of the nervous system is not associated with a lesion, and said step of defining said treatment protocol includes selecting compensating electromagnetic waves adapted for correcting non lesion associated deficits in said subjects.

It is further within the scope to provide a computer readable medium comprising instructions for implementing a protocol as defined in any of the above, comprising additional steps of transmitting a variety of electromagnetic waves of specified frequencies in a homogeneous field covering at least a portion of the volume of said at least a target portion of the nervous system of said subject which is resonated with said specified frequencies.

It is further within the scope to provide a computer readable medium comprising instructions for implementing a protocol as defined in any of the above, comprising an additional step of transmitting electromagnetic field frequencies in the range of between about 0.01 Hz (DC) to about 100 Hz.

It is further within the scope to provide a computer readable medium comprising instructions for implementing a protocol as defined in any of the above, comprising an additional step of transmitting electromagnetic field frequencies in intensity ranges from about 10̂-6 Gauss to about 100 Gauss.

It is further within the scope to provide a computer readable medium comprising instructions for implementing a protocol as defined in any of the above, comprising an additional step of producing EMF generated by at least one oscillating wave.

It is further within the scope to provide a computer readable medium comprising instructions for implementing a protocol as defined in any of the above, comprising an additional step of transmitting electromagnetic field frequencies characterized by pulse duration ranges from minutes to hours.

It is further within the scope to provide a computer readable medium comprising instructions for implementing a protocol as defined in any of the above, comprising an additional step of providing said treatment protocol that may vary from once per few days to several times a day. More particularly, the computer readable medium comprising instructions for implementing a protocol as defined in any of the above may include a step of providing at least one session of said treatment protocol at intervals selected from the group consisting of once a day, more than once per day, once per several days and any combination thereof.

It is further within the scope to provide a computer readable medium comprising instructions for implementing a protocol as defined in any of the above, comprising an additional step of providing said treatment protocol once a day for a period of days, weeks or months.

It is further within the scope to provide a computer readable medium comprising instructions for implementing a protocol as defined in any of the above, comprising an additional step of enhancing nerve fiber regeneration by increasing at least one nerve fiber parameter selected from the group consisting of size, length, thickness and any combination thereof.

It is further within the scope to provide a computer readable medium comprising instructions for implementing a protocol as defined in any of the above, comprising an additional step of inducing regeneration of damaged, injured or pathological, semi healthy or healthy nerve fibers or nerve fibers with a lesion or trauma.

It is further within the scope to provide a computer readable medium comprising instructions for implementing a protocol as defined in any of the above, comprising an additional step of decreasing the symptoms of at least one disease or syndrome or condition selected from the group consisting of: nervous system lesions or damage, brain injuries (trauma), stroke, heart transplant, movement syndromes, digestion syndromes, breathing syndromes, heart syndromes, spinal cord syndromes, spinal disc herniation.

It is further within the scope to provide a computer readable medium comprising instructions for implementing a protocol as defined in any of the above, comprising an additional step of defining said treatment protocol adapted for determining frequency, level and duration of the current supplied to said transmitter.

It is further within the scope to provide a computer readable medium comprising instructions for implementing a protocol as defined in any of the above, comprising an additional step of defining said treatment protocol adapted for determining frequency, level, duration, periodicities and any combination thereof of said compensating electromagnetic waves.

It is further within the scope to provide a computer readable medium comprising instructions for implementing a protocol as defined in any of the above, comprising an additional step of selecting said electromagnetic wave measuring device from the group consisting of: EEG, MEG, MRI, PET, any other acceptable monitoring or electromagnetic wave measuring means and any combination thereof.

It is further within the scope to provide a computer readable medium comprising instructions for implementing a protocol as defined in any of the above, comprising an additional step of configuring said measuring device for evaluating the results of the electromagnetic waves transmission.

It is further within the scope to provide a computer readable medium comprising instructions for implementing a protocol as defined in any of the above, comprising an additional step of evaluating said nerve fiber regeneration or neurogenesis by at least one parameter or test selected from the group consisting of relative leg placement test (FLP), relative front legs placement (FPS), relative neurological severity score (NSS), brain scans, nerve fiber scans, nerve fiber activity analysis, subventricular zone (SVZ) or subependymal zone or subgranular zone examination, MRI, PET, CT and any other available clinical test and any combination thereof.

It is further within the scope to provide a computer readable medium comprising instructions for implementing a protocol as defined in any of the above, comprising an additional step of configuring said apparatus for decreasing the recovery period after nerve fiber damage or trauma.

It is further within the scope to provide a computer readable medium comprising instructions for implementing a protocol as defined in any of the above, comprising an additional step of configuring said transmitter adapted for providing said electromagnetic wave useful for regenerating nerve fibers by means of inducing a resonance effect between the frequency profile of the transmitted electromagnetic wave and the profile of the natural tissue or region of interest of said subject.

It is further within the scope to provide a computer readable medium comprising instructions for implementing a protocol as defined in any of the above, comprising an additional step of defining said resonance effect by the enhanced activity detected by nerve fiber analysis.

It is further within the scope to provide a computer readable medium comprising instructions for implementing a protocol as defined in any of the above, comprising an additional step of transmitting said resonance effect thereby generating an electro-chemical transmission in the nerve cells and neuron of the selected nervous system portion.

It is further within the scope to provide a computer readable medium comprising instructions for implementing a protocol as defined in any of the above, comprising an additional step of providing said CPU further including a database comprising data useful for profiling the electromagnetic wave of said at least a target portion of the nervous system of said subject in order to adjust a treatment protocol to said at least a target portion of the nervous system of said subject.

It is further within the scope to provide a computer readable medium comprising instructions for implementing a protocol as defined in any of the above, comprising an additional step of configuring said CPU adapted to control said electromagnetic wave generator

It is further within the scope to provide a computer readable medium comprising instructions for implementing a protocol as defined in any of the above, comprising an additional step of configuring said CPU adapted to control said treatment periodicities.

It is further within the scope to provide a computer readable medium comprising instructions for implementing a protocol as defined in any of the above, comprising an additional step of defining said treatment protocol including a plurality of distinct frequencies within the range of the distinct tissue regions or target portions of the nervous system within said subject.

It is further within the scope to provide a computer readable medium comprising instructions for implementing a protocol as defined in any of the above, comprising an additional step of configuring said transmitter comprising at least one coil for generating peripheral electromagnetic field.

It is further within the scope to provide a computer readable medium comprising instructions for implementing a protocol as defined in any of the above, comprising an additional step of configuring said transmitter comprising at least one coil having a turn diameter sufficient to provide electromagnetic field to said target portion of the nervous system of said subject.

It is further within the scope to provide a computer readable medium comprising instructions for implementing a protocol as defined in any of the above, comprising an additional step of configuring said transmitter comprising at least one coil providing a uniform field to said at least a target portion of the nervous system of said subject while said target portion is oriented in any convenient alignment.

It is further within the scope to provide a computer readable medium comprising instructions for implementing a protocol as defined in any of the above, comprising an additional step of conforming said frequencies in accordance to specific tissues or body portions known to be capable of having an activity of interest.

It is further within the scope to provide a computer readable medium comprising instructions for implementing a protocol as defined in any of the above, comprising an additional step of transmitting said electromagnetic field in at least one selected frequency for at least one session.

It is further within the scope to provide a computer readable medium comprising instructions for implementing a protocol as defined in any of the above, comprising an additional step of synchronizing said transmission of said electromagnetic field with different nerve fiber types.

It is further within the scope to provide a computer readable medium comprising instructions for implementing a protocol as defined in any of the above, comprising an additional step of regenerating nerve fibers so as to thickening and/or elongating of existing nerve fibers or bundles, generation of the new nerve fiber and any combination thereof.

It is within the scope of the invention to provide an apparatus for regenerating nerve fibers in a subject. The apparatus essentially comprising: (a) at least one transmitter configured to generate an electromagnetic field (EMF) through at least a target portion of the nervous system of a subject; (b) at least one electromagnetic wave measuring device for detecting the electromagnetic wave profile of the at least a target portion of the nervous system of the subject; (c) at least one CPU for processing data concerned with detection of the electromagnetic wave profile of the at least a target portion of the nervous system of a subject, the CPU comprising a database for storing and analyzing wave profiles of normal patients and wave profiles of patients with nervous system lesions; and (d) at least one computer readable medium containing a predetermined protocol for operation of the transmitter; wherein the at least one CPU has a first module for selection of compensating electromagnetic waves for correcting anomalous wave profiles of the patients with the nervous system lesions; and a second module for processing data concerned with transmission of the compensating electromagnetic waves to the nervous system lesions and regions proximate to the lesions.

It is further within the scope to provide the apparatus as defined in any of the above, wherein the protocol comprises instructions to the transmitter for transmission of the compensating electromagnetic waves to provide a resonance effect; thereby inducing regeneration of nerve fibers at the nervous system lesions and regions proximate to the lesions.

It is further within the scope to provide the apparatus as defined in any of the above, wherein the transmitter is adapted to produce EMF in predetermined frequency and intensity.

It is further within the scope to provide the apparatus as defined in any of the above, configured to increase at least one nerve fiber parameter selected from the group consisting of size, length, thickness and any combination thereof.

It is further within the scope to provide the apparatus as defined in any of the above, configured to induce regeneration of damaged, injured or pathological, semi healthy or healthy nerve fibers or nerve fibers with a lesion or trauma.

It is further within the scope to provide the apparatus as defined in any of the above, wherein the transmitter produces EMF at frequency range of about 0.01 Hz (DC) to 100 Hz.

It is further within the scope to provide the apparatus as defined in any of the above, wherein the transmitter produces EMF generated by at least one oscillating wave.

It is further within the scope to provide the apparatus as defined in any of the above, wherein the electromagnetic wave is administered in the intensity of 10̂-6 to 100 Gauss.

It is further within the scope to provide the apparatus as defined in any of the above, wherein the electromagnetic wave is administered in the duration of minutes to about an hour.

It is further within the scope to provide the apparatus as defined in any of the above, wherein the apparatus is adapted to decrease the symptoms of at least one disease or syndrome or condition selected from the group consisting of: nervous system lesions or damage, brain injuries (trauma), stroke, heart transplant, movement syndromes, digestion syndromes, breathing syndromes, heart syndromes, spinal cord syndromes, spinal disc herniation.

It is further within the scope to provide the apparatus as defined in any of the above, wherein the predetermined protocol is adapted for determining frequency, level and duration of the current supplied to the transmitter.

It is further within the scope to provide the apparatus as defined in any of the above, wherein the predetermined protocol is adapted for determining frequency, level, duration, periodicities and any combination thereof of the electromagnetic waves.

It is further within the scope to provide the apparatus as defined in any of the above, wherein the electromagnetic wave measuring device is selected from the group consisting of: EEG, MEG, MRI, PET or any other acceptable monitoring or measuring means and any combination thereof.

It is further within the scope to provide the apparatus as defined in any of the above, wherein the measuring device is configured for evaluating the results of the electromagnetic waves transmission.

It is further within the scope to provide the apparatus as defined in any of the above, wherein the nerve fiber regeneration or neurogenesis is evaluated by at least one parameter, test or scanning or imaging device selected from the group consisting of relative leg placement test (FLP), relative front legs placement (FPS), relative neurological severity score (NSS), brain scans, nerve fiber scans, nerve fiber activity analysis, subventricular zone (SVZ) or subependymal zone or subgranular zone examination, MRI, PET, CT and any other available clinical test and any combination thereof.

It is further within the scope to provide the apparatus as defined in any of the above, configured for decreasing the recovery period after nerve fiber damage or trauma.

It is further within the scope to provide the apparatus as defined in any of the above, wherein the transmitter is adapted for providing the electromagnetic wave useful for regenerating nerve fibers by means of inducing a resonance effect between the frequency profile of the transmitted electromagnetic wave and the wave frequency profile of the natural tissue or region of interest of the subject.

It is further within the scope to provide the apparatus as defined in any of the above, wherein the resonance effect is defined by the enhanced activity detected by nerve fiber analysis.

It is further within the scope to provide the apparatus as defined in any of the above, wherein the resonance effect generates an electro-chemical transmission in the nerve cells and/or neuron of the determined nervous system.

It is further within the scope to provide the apparatus as defined in any of the above, wherein the CPU further includes a database comprising data useful for profiling the electromagnetic wave of the at least a target portion of the nervous system of the subject in order to adjust a treatment protocol to the at least a target portion of the nervous system of the subject.

It is further within the scope to provide the apparatus as defined in any of the above, wherein the CPU is further adapted to control the electromagnetic wave generator.

It is further within the scope to provide the apparatus as defined in any of the above, wherein the CPU is further adapted to control the treatment periodicities.

It is further within the scope to provide the apparatus as defined in any of the above, wherein the predetermined protocol includes a plurality of distinct frequencies within the range of the distinct tissue regions or target portions of the nervous system within the subject.

It is further within the scope to provide the apparatus as defined in any of the above, wherein the transmitter comprises at least one coil for generating peripheral electromagnetic field.

It is further within the scope to provide the apparatus as defined in any of the above, wherein the at least one coil is a Helmholtz coil, having a turn diameter sufficient to provide electromagnetic field to the target portion of the nervous system of the subject.

It is further within the scope to provide the apparatus as defined in any of the above, wherein the apparatus comprises at least one coil providing a uniform field to the at least a target portion of the nervous system of the subject while the target portion is oriented in any convenient alignment.

It is further within the scope to provide the apparatus as defined in any of the above, wherein the frequencies are conformed in accordance to specific tissues or body portions known to be capable of having an activity of interest.

It is further within the scope to provide the apparatus as defined in any of the above, wherein the electromagnetic field is transmitted in at least one selected frequency for at least one session.

It is further within the scope to provide the apparatus as defined in any of the above, wherein the transmission of the electromagnetic field is synchronized with different nerve fiber types.

It is further within the scope to provide the apparatus as defined in any of the above, wherein the nerve regeneration procedure results in the thickening and/or elongation of existing nerve fibers or bundles, generation of the new nerve fibers and any combination thereof.

It is further within the scope to provide a method for regenerating nerve fibers of a subject comprising the steps of: (a) selecting at least a target portion of the nervous system of a subject; (b) providing the apparatus as defined in any of the above; (c) detecting electromagnetic wave profile of the at least a target portion of the nervous system of the subject; (d) analyzing wave profiles of normal patients and wave profiles of patients with nervous system lesions; and (e) defining a treatment protocol, wherein the step of defining the treatment protocol includes selecting compensating electromagnetic waves adapted for correcting anomalous wave profiles of the patients with the nervous system lesions; and transmitting to the subject the protocol of compensating electromagnetic waves to provide a resonance effect; thereby inducing regeneration of nerve fibers at the nervous system lesions and regions proximate to the lesions.

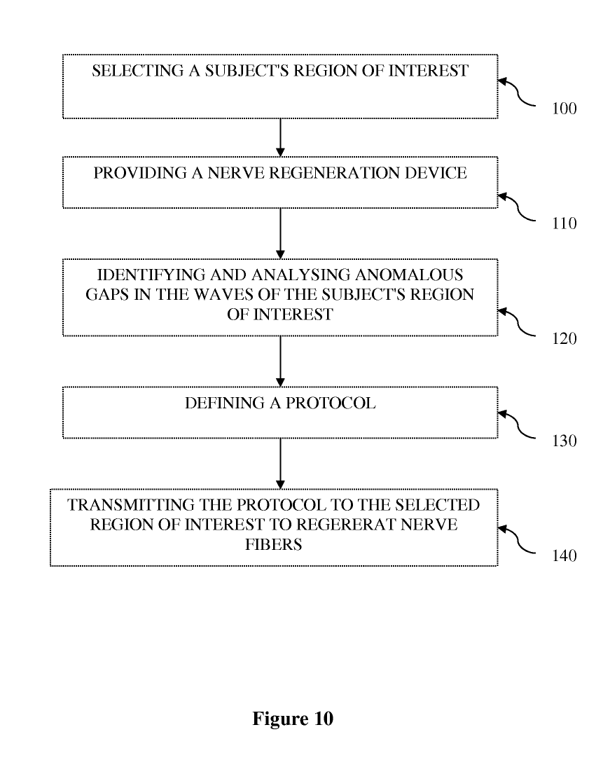

Reference is now made to FIG. 10 presenting a schematic diagram of the method for regenerating nerve fibers of a subject, in accordance with an embodiment of the present invention. The method comprising the step of: (a) selecting a nervous system region of interest of a subject, (b) providing an apparatus for regenerating nerve fibers of a subject; (b) identifying and analyzing anomalous gaps in the nervous system wave profile of a subject; (d) defining a treatment protocol, (e) transmitting to the subject nervous system the protocol, wherein the step of defining the protocol includes providing an electromagnetic frequency profile including compensatory waves; further wherein the protocol provides a resonance effect thereby inducing nerve fiber regeneration in a tissue area having the pathology or lesion of interest or in the corresponding healthy tissue of interest.

There is further provided in accordance with a preferred embodiment of the present invention the method as defined above, further comprising the step of identifying a site of the pathological condition in the body of the subject, and wherein the step of selecting the body region comprises selecting a neural region of the body remote from the pathological site.

In order to understand the invention and to see how it may be implemented in practice, a plurality of preferred embodiments will now be described, by way of non-limiting example only, with reference to the following examples.

### Example 1

The method and apparatus were demonstrated on rats. The rats were subjected to transient (tMCAO) occlusion of the middle cerebral artery (MCAO) stroke procedure, then they were divided to control and treatment groups. The rats were treated by electromagnetic field at different frequencies, denoted as Induction A: about 3.93 Hz, Induction B: about 15.72 Hz, Induction C: about 7.86 Hz and Induction D: about 31.44 Hz. The treatment and control groups were then subjected to various clinical tests and measurements including Leg placement (FLP) and the general assessment (relative neurological severity score, NSS) and/or to scanning and imaging measurements.

An exemplary experimental protocol would be subjecting the rats to stroke on day 1, then subjecting the treatment groups rats to EMF (electro-magnetic field) for four to eight weeks, once a day for 8 min in various frequencies (i.e. see above frequencies). The treatment protocol was preferably performed using the device of the present invention.

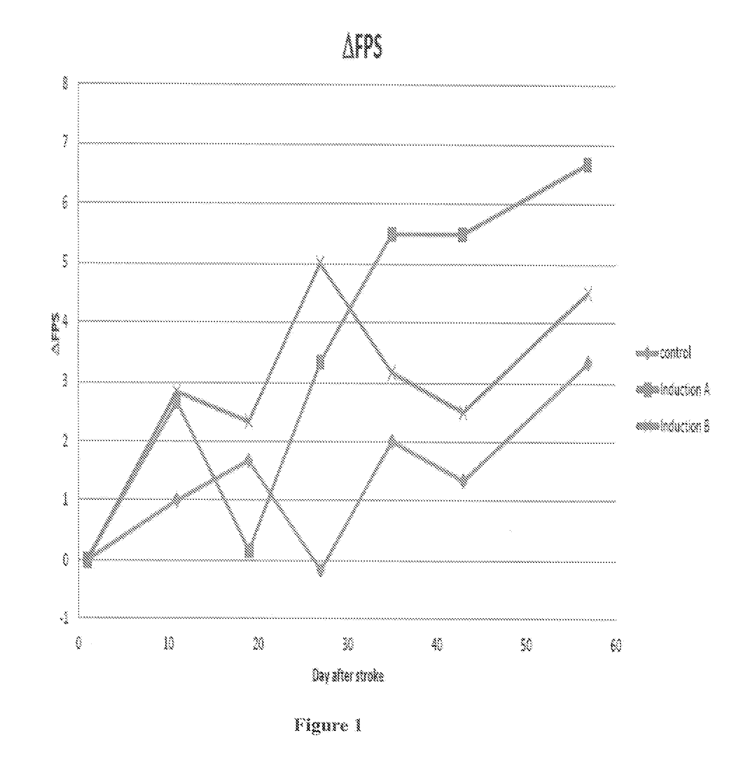

Reference is now made to FIG. 1 presenting a graphic representation of relative Leg placement Forward Leg Position (FLP) or FPS measurements during stroke recovery, in control and treatment groups. As can be seen from this figure, the recovery performance of the treatment groups (Induction A and Induction B) was significantly enhanced relative to the control group. The treatment groups, Induction A and Induction B, exhibited enhanced recovery rate as compared to the control group and further reached a higher FPS improvement level, 55 days after the stroke. It is further shown in this figure that treatment with 3.93 Hz protocol (Induction A) provides a higher FPS recovery level as compared to Induction B and control groups.

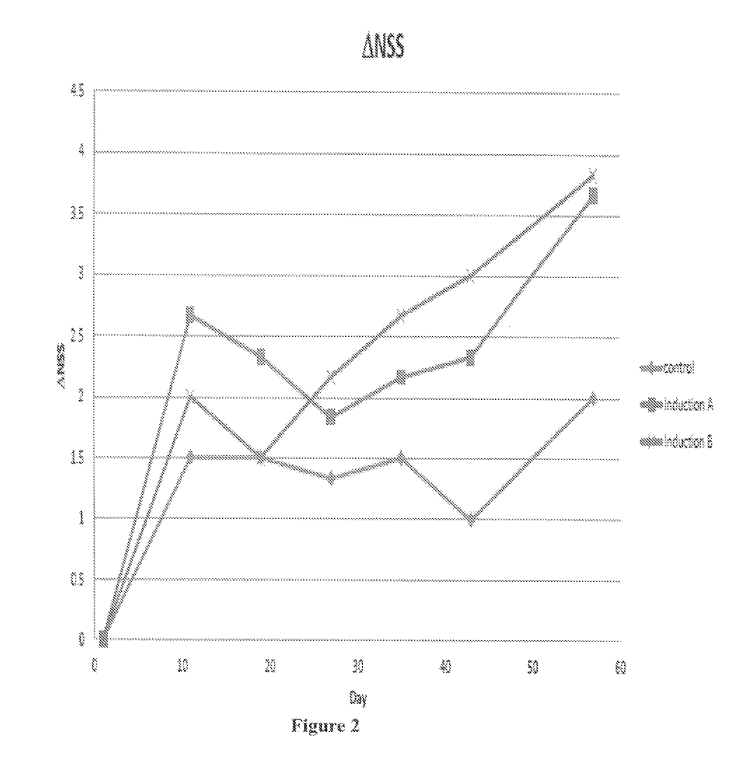

Reference is now made to FIG. 2 presenting a graphic representation of relative neurological severity score (NSS) measurements during stroke recovery, in control and treatment groups. The results shown in this figure clearly show that the rats treated by the apparatus and protocol of the present invention (Induction A and Induction B groups) exhibit a significantly higher recovery rate and level with respect to neurological severity score (NSS) parameter, as compared to the untreated rats (control group).

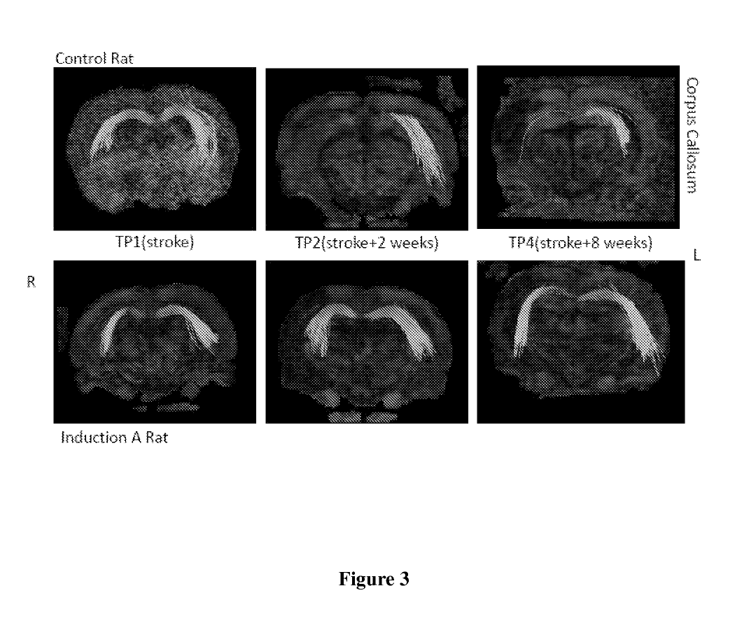

Reference is now made to FIG. 3 presenting MRI images of the subventricular zone (SVZ) of the brain during stroke recovery in treated rats as compared to control rats. It is demonstrated by the MRI scans that while the size of the SVZ increases dramatically after stroke in the rats belonging to the control group (see treatment periods TP1 and TP4), the SVZ does not increase its size in the treated groups (Induction A and Induction B groups) during treatment periods 1 to 4 (see TP1, defined as the day after stroke, as compared to TP4, defined as two months after stroke and one month after EMF treatment was completed). Thus it can be concluded by the results of the present invention that the effect of stroke is reduced dramatically by using the apparatus and protocols as inter alia disclosed. Moreover, it can be seen in this figure that in the treatment groups, in at least two sections of the brain, the stroke affected tissue region reaches its normal tissue color (darkens) with time during the treatment recovery periods.

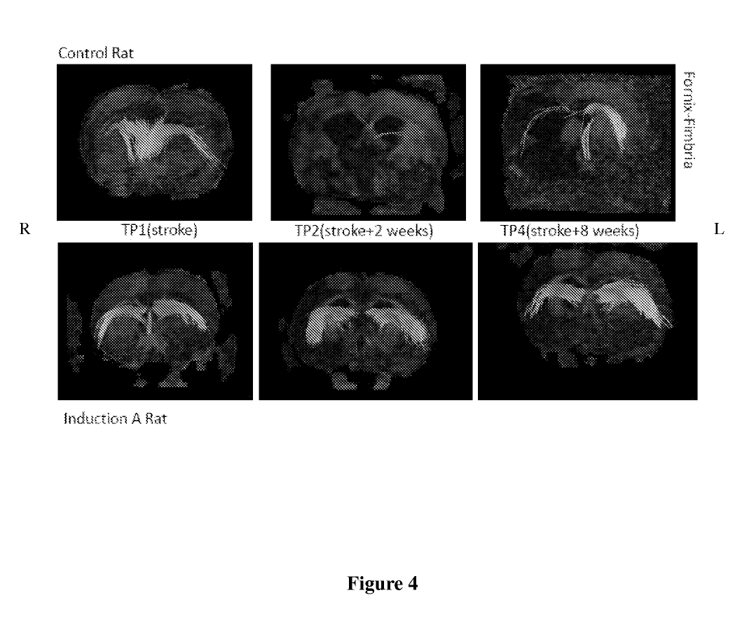

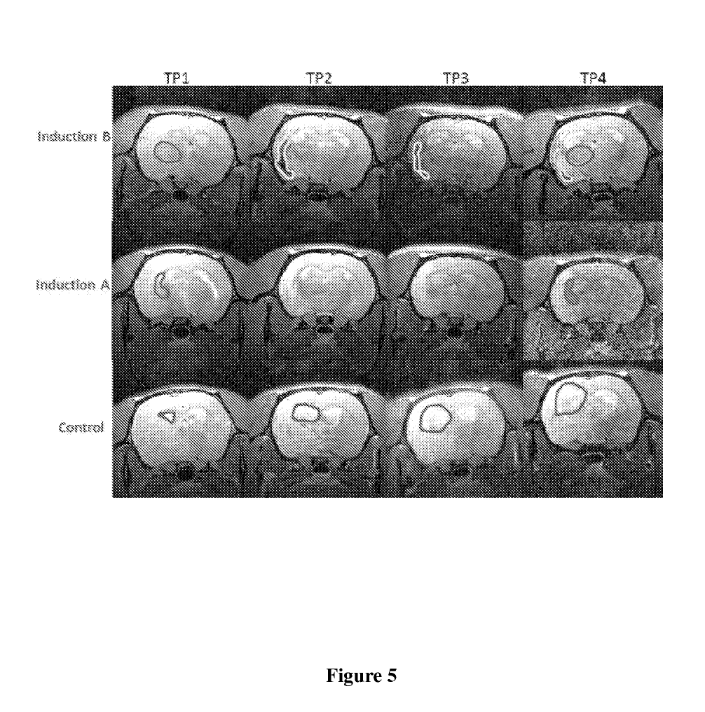

Reference is now made to FIG. 4 presenting nerve fiber images of the corpus callosum after stroke procedure in treated rats as compared to control rats. The rats in this experiment were exposed to stroke procedure on the right side of the brain, while the left part of the brain was not affected. As can be seen, unlike the control rats, where the fibers have died and did not recover with time (see TP1 to TP4 of FIG. 5), the treated rats (Induction A) have shown significant recovery during the treatments periods TP1 to TP4 and regain most of their fibers. In fact it is clearly demonstrated that by treating the brain with the apparatus and protocols of the present invention, the nerve fibers of the affected part of the brain regenerate and reaches normal level already two weeks after exposure to the stroke procedure. The results presented in FIG. 5 further show that as a results of the herein disclosed treatment, the brain fibers are increased, not only in the damaged part of the brain but also in the healthy part of the brain, demonstrating that the device and treatment provided by the present invention is valid to healthy or semi healthy organs as well.

Thus in the normal stroke scenario, the nerve cells die and the fibers connecting them to other brain cells die as well, in a time period of a few days (control, TP2, defined as two weeks after stroke and two weeks into the EMF treatment period, of FIG. 4). The nerve fibers begin to regenerate only 8 weeks after stroke procedure. However, it is shown by the experimental results herein provided that exposing the rats to EMF in the specific frequency ranges and duration, not only decrease or even eliminate the undesired effect, it is also unexpectedly regenerate new fibers both in the region of the stroke (Induction A, right side −R of FIG. 4), and also regenerate new fibers even in the regions not affected by stroke (Induction A, left side-L of FIG. 4).

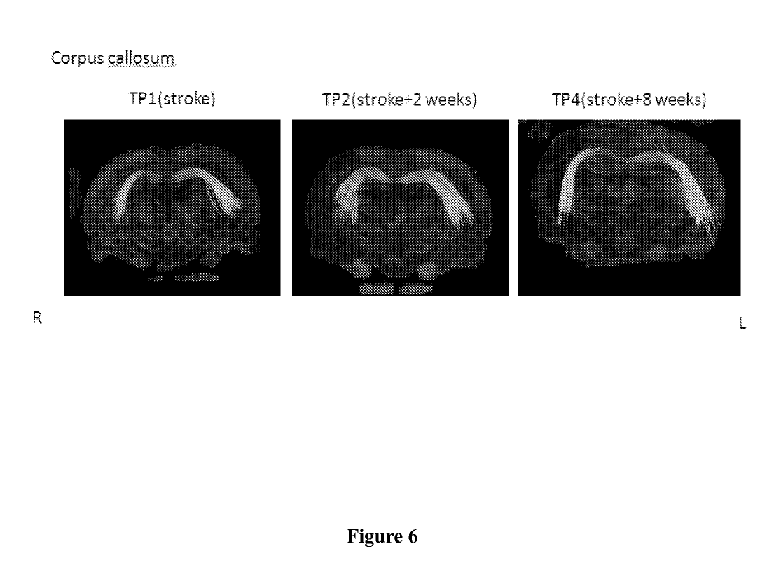

Reference is now made to FIG. 5 presenting images of the Fronix-Fimbria after stroke procedure in treated rats as compared to control rats. The results presented in this figure strengthen the results and conclusions presented in FIG. 5. It is again demonstrated in FIG. 6 that in the control rats, two weeks after stroke, the nerve cells die and the fibers connecting them to other brain cells die as well (control rats TP2). Low level of nerve regeneration begins to be exhibited in the affected region only after 8 weeks from the stroke procedure (control rats TP4). However, in the treated rats, dramatic induction of nerve fiber regeneration in the affected region is exhibited already after 2 weeks from stroke (Induction A, TP2). Nerve fiber regeneration is further exhibited in the non affected part of the brain exposed to the treatment protocol of the present invention (Induction A, TP2 and TP4).

### Example 2

Reference is now made to FIGS. 6-9 presenting images of different parts of the brain of rats subjected to stroke procedure as described above. The rats were subjected to stroke procedure on the right side of the brain (R), while the left side (L) is unaffected. During stroke recovery period, the rats were exposed to treatment protocols as defined above.

Reference is now made to FIG. 6 presenting images of the Corpus callosum after stroke procedure in accordance with an embodiment of the present invention. As shown in this figure, nerve fiber degradation is exhibited in the affected side (R) of the Corpus callosum (see TP1). After two weeks (TP2), thickness of the fiber increases and after 8 weeks (TP4) fibers of both sides of the Corpus callosum, namely, the affected (R) non affected (L) are nearly symmetrical. It is further demonstrated in this figure that by using the method and apparatus of the present invention, regeneration of nerve fibers of the healthy part of the brain (L) is achieved.

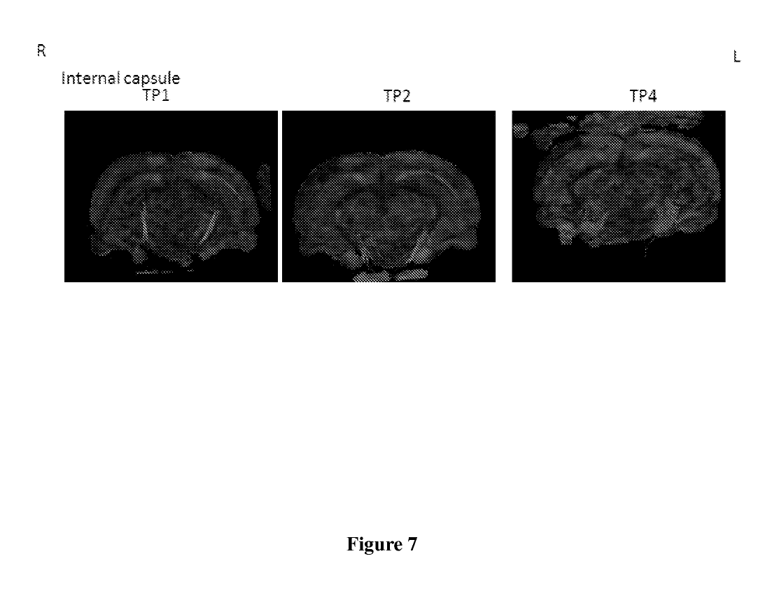

Similar results and therapeutic effects are demonstrated in FIG. 7 which presents images of the internal capsule. This figure shows nerve fiber regeneration already two weeks (TP2) after stroke procedure in accordance with an embodiment of the present invention. The nerve fiber enhancement is demonstrated both in the affected part of the brain (R, TP2 and TP4) and in the healthy part of the brain (L, TP4).

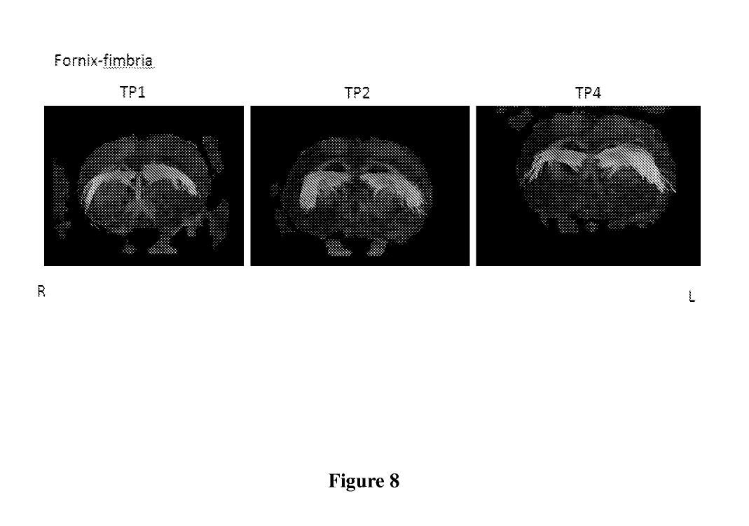

Reference is now made to FIG. 8 presenting images of the Fornix fimbria during stroke recovery. It is shown that as a result of the stroke procedure, nerve fiber degradation and significant decrease in fiber thickness were observed (R, TP1), as compared to the healthy part of the brain (L, TP1). After two weeks, thickness of the nerve fiber is increased (TP2), and after 8 weeks, fibers of both the affected and healthy regions of the brain, have thickened and show a symmetry in shape.

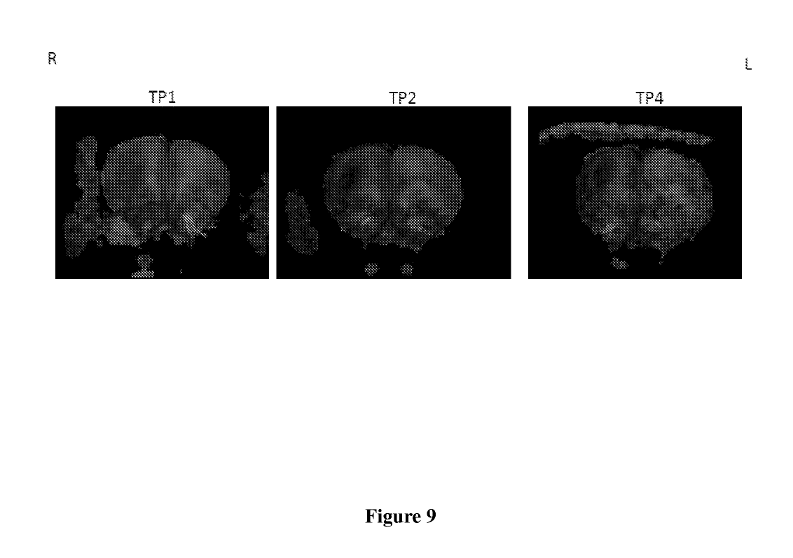

Reference is now made to FIG. 9 presenting images of the anterior commissure, demonstrating nerve fiber regeneration after stroke procedure, similarly to the results shown in FIG. 8.

The experiments above show that after stroke procedure, nerve fiber degradation and/or significant decrease in fiber thickness is exhibited in the affected side of the brain (TP1). After two weeks (TP2), the thickness of the fiber increases and after 8 weeks (TP4), fibers of both sides of the brain, namely, the affected (R) non affected (L) parts are nearly symmetrical in their thickness and shape. Thus the apparatus and method of the present invention are herein demonstrated to increase nerve fiber regeneration both in the injured or damaged nerve fibers as well as in the non affected or healthy corresponding parts of the nervous system.

Thus, in view of the above results it can be concluded that after the injury or trauma (i.e. stroke), the nerve cells either die or the nerve fibers are both decrease in length and in thickness. During recovery, the nerve fibers increase in thickness and length and they re-gain somewhat symmetrical appearance to the other, non effected nerve fibers within the corresponding tissue. It is herein emphasized that by using the disclosed device and method, newly generated nerve fibers and/or increase in nerve fibers length, thickness and size is achieved in selected portions of the nervous system.

1. A method for regenerating nerve fibers of a subject, the method comprising:
selecting at least a target portion of the nervous system of a subject with a lesion in the nervous system;
detecting using an electromagnetic wave measuring device to measure an electromagnetic wave profile of said at least a target portion of said nervous system of said subject;
comparing the detected wave profiles of said subject with wave profiles of normal patients;
defining a treatment protocol by selecting compensating electromagnetic waves adapted for correcting anomalous wave profiles associated with said lesion; and
transmitting to said subject said compensating electromagnetic waves to provide a resonance effect to induce regeneration of nerve fibers at said lesion and regions proximate to said lesion.

2. (canceled)

3. The method of claim 1, wherein transmitting to said subject comprises transmitting said compensating electromagnetic waves of specified frequencies in a homogeneous field covering at least a portion of the volume of said at least a target portion which is resonated with said specified frequencies.

4-12. (canceled)

13. The method of claim 1, wherein defining said treatment protocol comprises determining a frequency, level and duration of current that is supplied to a transmitter.

14. The method of claim 1, wherein defining said treatment protocol comprises determining a frequency, level, duration, and periodicities of said compensating electromagnetic waves.

15. The method of claim 1, wherein the electromagnetic wave measuring device is configured to apply a measurement technique selected from a group of techniques consisting of: EEG, MEG, MRI, and PET.

16. The method of claim 1, additionally comprising using said electromagnetic wave measuring device to evaluate results of the electromagnetic waves transmission.

17. The method of claim 16, wherein the evaluation of the results comprises an evaluation selected from a group of nerve fiber regeneration evaluations consisting of relative leg placement test (FLP), relative front legs placement (FPS), relative neurological severity score (NSS), brain scans, nerve fiber scans, nerve fiber activity analysis, subventricular zone (SVZ) examination, subependymal zone examination, subgranular zone examination, MRI, PET, and CT.

18-25. (canceled)

26. The method of claim 1, wherein transmitting to said subject said compensating electromagnetic waves comprises generating a peripheral electromagnetic field.

27-96. (canceled)
